(19) United States
(12) Patent Application Publication
(10) Pub. No.: US 2026/0133176 A1
(43) Pub. Date: May 14, 2026

(54) DIGITAL ANALYSIS OF HETEROGENEOUS MATERIALS

(71) Applicant: SIKA TECHNOLOGY AG, Baar (CH)

(72) Inventors: Ulf VELTEN, Oetwil an der Limmat (CH); Annette WEINKAUF, Staufen (CH)

(73) Assignee: SIKA TECHNOLOGY AG, Baar (CH)

(21) Appl. No.: 19/119,178

(22) PCT Filed: Dec. 7, 2023
(86) PCT No.: PCT/EP2023/084814

§ 371 Date: Apr. 8, 2025

(30) Foreign Application Priority Data
Dec. 7, 2022 (WO) PCT/EP2022/084816

(51) Int. Cl. G01N 33/38; G01N 15/075; G01N 15/1433; G06T 7/00; G06T 7/40; G06T 7/62; G06T 7/70; G06T 7/90; G16C 60/00; H04N 23/60

(52) U.S. Cl. CPC G01N 33/383; G01N 15/075; G01N 15/1433; G06T 7/0002; G06T 7/40; G06T 7/62; G06T 7/70; G06T 7/90; G16C 60/00; H04N 23/64; G06T 2207/10024

(57) ABSTRACT

A computer-implemented method, includes steps: providing or selecting a sample of heterogeneous material to be analyzed; b) taking at least one digital image of the sample with a camera of a computer device, mobile computer device, or camera connected to a computer device, in which at least one digital image that was pre-recorded with standalone camera; c) performing image analysis, of at least one digital image extracting following properties: a size parameter, shape parameter, particle shape parameter, spatial distribution, orientation parameter, surface parameter, particle surface parameter, roughness parameter, packing density, segregation parameter, color, and/or an area share of constituents, particle-shaped constituents, identified by image analysis in at least one digital image; and/or an area share of continuous phase identified by image analysis in at least one digital image; and d) making available one or more properties extracted in step c) via user interface, machine interface and/or data storage medium.

### TECHNICAL FIELD

The invention relates to a computer-implemented method for characterization of a heterogeneous material comprising distributed constituents, especially distributed particle-shaped constituents, dispersed within a continuous phase of condensed matter, as well as systems with means to carry out the method, and a computer-readable medium comprising instructions for carrying out the method.

### BACKGROUND ART

In construction industry, building materials based on hardenable or hardened compositions are widely used for various applications. Examples of such compositions are mortar, concrete, grout, screed, adhesive, flooring or coating compositions. These compositions typically comprise hardenable binders, optionally in combination with water, air, solid aggregates and/or additives.

Thereby, binders might be selected from mineral binders, such as e.g. hydraulic binders, as well as organic binders, e.g. curable polymers. Aggregates are chosen depending on the desired properties of the curable compositions. Typically aggregates include sand, gravel, rock flour, metallic particles and/or polymeric particles. Air might be introduced to produce lightweight structures or to obtain specific properties, such as e.g. freeze-thaw resistance.

Hardenable or hardened compositions as used in construction industry usually are heterogeneous materials comprising two or more different constituents. Thereby, in particular, distributed particle-shaped constituents, e.g. aggregates, air voids and/or polymeric particles, are dispersed within a continuous phase of condensed matter, e.g. within a solvent, a fluid binder material or a hardened binder in solid state.

In order to ensure that the hardenable compositions meet desired requirements during processing and later on in hardened state, various testing procedures have been developed.

For example, raw materials of the hardenable compositions such as e.g. aggregates and fillers usually are checked in terms of particle size distribution in order to ensure proper grading curves. Also, it is known to analyze particle shapes of aggregates and contamination of recycled aggregates to adjust the mix design of mortar or concrete compositions.

In this regard, GB 2524130 (LPW Technology Ltd) describes for example an analyzing system for determining the characteristics and/or properties of a flowable material (e.g. sand) comprising an attachable analyzing apparatus and a smartphone or tablet device. The sample material in the sample container moves through the flow aperture into the analyzing chamber where the smartphone or tablet device captures images of the sample material. An application installed on the smartphone is configured for analyzing the sample by determining size, shape, colour, flow rate and speed of the sample particles.

Furthermore, workability and consistence of fresh mineral binder compositions can be checked for example with well-known slump flow tests, flow table tests, L-box tests, V-funnel tests and the like. In hardened state, the compositions usually are analyzed in terms of density, compressive strength, flexural strength, tensile strength, Young's modulus, fracture patterns, water tightness, surface aesthetics, air voids, and many more properties.

Although many analytical methods are available to characterize hardenable or hardened compositions, most of these methods, with few exceptions, are time consuming, require sophisticated equipment and are phenomenologically oriented. Likewise, other building materials, such as e.g. membranes for roofing as well as foams for installation, sealing, reinforcing, damping and/or filling purposes, which are for example used in automotive industry, need to be inspected with regard to their condition to ensure quality requirements or to assess their condition after a certain exposure, especially when it comes to crack formation. There are various test methods for such inspections, but these are also usually elaborate and time consuming.

Therefore, there is still a need for improved solutions that have fewer or none of the aforementioned disadvantages.

### Disclosure of the Invention

It is an object of the present invention to provide improved solutions for analyzing heterogeneous materials comprising distributed constituents, especially distributed particle-shaped constituents, dispersed within a continuous phase of condensed matter. Especially, the solutions should allow for analyzing heterogeneous materials without need for sophisticated equipment in a manner as easy, flexible and user friendly as possible.

Surprisingly, it has been found that the features of claim 1 achieve this object. Thus, the core of the present invention is related to a computer-implemented method for characterization of a heterogeneous material comprising distributed constituents, especially distributed particle-shaped constituents, dispersed within a continuous phase of condensed matter, comprising the steps of:
a) Providing or selecting a sample of the heterogeneous material to be analyzed;
b) Taking at least one digital image of the sample with a camera of a computer device, especially a mobile computer device, or a camera connected to a computer device, especially a mobile device and/or with the computer device read in at least one digital image that was pre-recorded with a standalone camera;
c) Performing an image analysis, especially an imaging particle analysis, of the at least one digital image for extracting one or more of the following properties:
   a size parameter, especially a particle size parameter, a shape parameter, especially a particle shape parameter, a spatial distribution, an orientation parameter, especially a particle orientation parameter, a surface parameter, especially a particle surface parameter, a roughness parameter, especially a particle roughness parameter, a packing density, a segregation parameter, a color, and/or an area share of the constituents, especially the particle-shaped constituents, identified by the image analysis in the at least one digital image; and/or
   an area share of the continuous phase identified by the image analysis in the at least one digital image;
d) Making available the one or more of the properties extracted in step c) via a user interface, via a machine interface and/or on a data storage medium.

The inventive method is a unique approach, which can be implemented on conventional mobile devices, such as e.g. smartphones. Thus, there is no need for complex and expensive analysis equipment. The method furthermore allows for a very fast, flexible and accurate digital characterization of heterogeneous materials, in terms of various properties.

Thereby, heterogeneous materials of different nature and structure can easily be characterized with one and the same hardware device. For example, heterogeneous material in the form of emulsions, foams, suspensions, mineral and/or organic binder compositions in fluid as well as in hardened state can be analyzed without need for special equipment. Thereby, hardened heterogeneous materials may e.g. be obtained by chemically and/or physically curing fluid heterogeneous materials.

In the present context, the term foam is to be understood in a broad sense and in particular encompass solid as well as liquid foams made of essentially any kind of material. For example, foams can be in the form of foamed mineral and/or organic binder compositions, foamed polymeric materials, foamed liquids and the like.

As an example, foamed polymeric materials can be obtained from thermally expandable compositions comprising a polymer matrix with one or more polymers and a chemical or physical blowing agent. Such compositions can be used, for example, as baffles and acoustic damping materials in automotive industry.

Therefore, the inventive method is in particular suitable for characterizing hardenable or hardened compositions used in construction industry, such as e.g. mortars, concretes, grouts, screeds, flooring compositions, adhesives, coating compositions, or foams, both in workable as well as in hardened state.

Likewise, synthetic materials, such as e.g. synthetic membranes used for waterproofing and/or roofing, can be characterized, in particular synthetic materials comprising air voids and/or cracks.

In particular, the inventive method allows for characterization of the heterogeneous materials on a structural level. In particular, size parameters, shape parameters, spatial distributions, segregation parameters, colors, and/or area shares of the constituents, as well as the area share of the continuous phase, can directly be obtained. Specifically, particle size parameters, particle shape parameters, spatial distributions, segregation parameters, colors, and/or area shares of the particle-shaped constituents, as well as the area share of the continuous phase, can directly be obtained. These parameters have a decisive impact on the properties of the heterogeneous materials so that for many materials it is possible to replace common testing procedures with the inventive method.

Additionally, the inventive method allows for storing the extracted data, e.g. on a remote server, for further use. For example, the extracted data might be combined and/or correlated with further data such as e.g. raw material data, mix design data of the heterogeneous materials, physical and/or chemical properties of the heterogeneous materials, to gain further insight into the relations between structure and properties of the heterogeneous materials.

Thanks to the fast and easy way to carry out characterization of the heterogeneous materials in particular with a mobile computer device, the inventive method can be implemented in a highly user-friendly manner.

Specifically, the method can be implemented in various ways, e.g. in the form of a standalone application running on the mobile device without need for any further resources such as for example server systems. This is in particular helpful in areas with limited access to communication networks, e.g. far away from urban centers or underground. However, the method can be implemented as well in a distributed computing environment, e.g. comprising the mobile device as a client in combination with a dedicated server as a storage unit and/or with a specialized processing unit.

Also, the inventive method can be implemented in a flexible manner with known software architectures, e.g. as native application, a progressive web application (PWA), or a hybrid application (combination of native and PWA). Thereby, helpful internet links to tutorials or support sites as well as sharing functions (e.g. via e-mail, Bluetooth, Airdrop, or others communication means) can be included in such applications as well. Also, the inventive method can be implemented in one single application, or it may be divided into two or more separate applications with appropriate software interfaces for data exchange between the applications. Furthermore, the application(s) can be extended with additional functions in a flexible manner.

Applications can be implemented for any kind of operating systems such as e.g. IOS, Android, Microsoft Windows and/or Linux.

The applications can be made available easily for anyone via different distribution channels, such as e.g. public download centers (for example Apple® Store and Google Play®) private company operated websites and/or dedicated download sites.

Additional aspects of the invention are subject of further independent claims. Particularly preferred embodiments are outlined throughout the description and the dependent claims.

### Ways of Carrying Out the Invention

A first aspect of the present invention is directed to a computer-implemented method for characterization of a heterogeneous material comprising distributed constituents, especially particle-shaped constituents, dispersed within a continuous phase of condensed matter, comprising the steps of:
a) Providing or selecting a sample of the heterogeneous material to be analyzed;
b) Taking at least one digital image of the sample with a camera of a computer device, especially a mobile computer device, a camera connected to a computer device, especially a mobile computer device, and/or with the computer device read in the at least one digital image that was pre-recorded with an external camera;
c) Performing an image analysis, especially an imaging particle analysis, of the at least one digital image for extracting one or more of the following properties:
   a size parameter, especially a particle size parameter, a shape parameter, especially a particle shape parameter, a spatial distribution, an orientation parameter, especially a particle orientation parameter, a surface parameter, especially a particle surface parameter, a roughness parameter, especially a particle roughness parameter, a packing density, a segregation parameter, a color, and/or an area share of the constituents, especially the particle-shaped constituents, identified by the image analysis in the at least one digital image; and/or
   an area share of the continuous phase identified by the image analysis in the at least one digital image;
d) Making available the one or more of the properties extracted in step c) via a user interface, via a machine interface and/or on a data storage medium.

Especially the computer device, in particular the mobile computer device, comprises a human interface device, especially comprising an input device and a display, and, preferably a communication interface, most preferably a wireless communication interface.

In the present context, a mobile computer device in particular is meant to be a handheld computer, i.e. a computer small enough to hold and operate in the hand of a human.

The mobile computer device in particular is selected from a mobile phone, a mobile computer or a portable computer. Especially, the mobile computer device is selected from a smartphone, a phablet, a tablet computer, a portable computer, a smartwatch, and/or a head-mounted display with camera. Highly preferred are mobile phones and/or smartphones.

Mobile phones and smartphones typically are equipped with high resolution cameras, an input device and a display. Thereby the input device and the display typically are combined in a touch sensitive display. Therefore, mobile phones and smartphones provide all of the hardware components required for performing the inventive method. Furthermore, such kind of devices can be held stably in the hand or be mounted on a mobile stand and/or tripod, which makes them highly suitable for taking images. At the same time mobile phones and smartphones typically have displays that are large enough for displaying complex data in well readable manner.

The expression “a camera of a (mobile) computer device” is meant to be an internal camera, which is integrated in the (mobile) computer device. In contrast, “a camera connected to a (mobile) computer device” means an external camera, which is a separate device connected to the (mobile) computer device. For example, the external camera is a camera attachable to the (mobile) computer device or a standalone camera. The connection between the external camera and the (mobile) computer device can be a wired and/or a wireless connection.

Alternatively or in addition, it is possible to pre-record the at least one digital image with a standalone camera and read in the image with the (mobile) computer device. Thereby, in particular, the standalone camera has an own memory device for intermediate storage of the image. Later on the at least one digital image can be transferred from the memory device to the (mobile) computer device in any manner known to the skilled person, e.g. by a wired connection, a wireless connection, and/or a physical exchange of the memory device.

Preferably, the camera is a camera for taking images in the visible spectrum, especially color images. Color images allow for considering color properties of the constituents, especially the particle-shaped constituents, and/or continuous phase in step c) of the inventive method. Thus, preferably, the at least one digital image taken in step b) is a color image. However, if color information is not required, other cameras, e.g. monochromatic cameras, can be used as well. In this case, the image is a monochromatic image, e.g. a black-and-white image.

Also, it is possible to use a camera for taking images outside the visible spectrum, e.g. in the infrared and/or ultraviolet range of the electromagnetic spectrum. Such cameras can be used alternatively or in addition to other cameras. In this case the properties of the constituents, especially the particle-shaped constituents, and/or the continuous phase in spectral ranges outside the visible spectrum can be considered in step c).

Preferably, the camera has a resolution of at least 2 megapixels, especially at least 5 megapixels, preferably at least 8 megapixels, particularly at least 12 megapixels, highly preferred at least 20 megapixels, even more preferred at least 50 megapixels or at least more than 100 megapixels. Especially, the camera has a resolution of at least 3′800 pixels×at least 2′100 pixels or a so called 4K, preferably 8K, more preferably 12K, especially 16K resolution. The higher the resolution the smaller constituents, especially particle-shaped constituents, can be identified. However, for special embodiments, cameras with lower resolutions might be suitable as well.

Especially, the computer device, especially the mobile computer device, is configured for automatically recognizing the camera resolution.

Especially, a size of the optical sensor of the camera is at least ⅓″, in particular at least 1/2.5″, preferably at least 1/1.7″, preferably at least ⅔″, particularly at least 1/1.33″ or at least 1/1.2″. Particularly, a size of the optical sensor of the camera is from ⅓″ to 1″.

Put differently, a size of the optical sensor of the camera in terms of width×height preferably is at least 4.8 mm×3.6 mm, in particular at least 5.7 mm×4.2 mm, preferably at least 7.6 mm×5.7 mm, preferably at least 8.8 mm×6.6 mm, particularly at least 9.6 mm×7.2 mm or at least 10.6 mm×8.0 mm. Particularly, a size of the optical sensor of the camera in terms of width x height is from 4.8 mm×3.6 mm to 13.2 mm×8.8 mm.

In general, the larger the sensor size, the more light can be captured by the sensor, which in turn improves the image quality. However, cameras with other sensor sizes might be suitable as well.

Additionally, supplementary lenses might be added to the camera, e.g. for extending or shortening the focal length of the camera.

Also, if available, additional internal wide angle and/or magnification lenses, both optical and/or digital, of the computer device, especially the mobile computer device, can be accesses on demand for taking the at least one digital image.

In step b), the at least one digital image preferably is taken from a surface, especially a flat surface, of the sample of the heterogeneous material. Thereby, preferably, the surface is oriented vertically or horizontally, especially preferred horizontally.

In further preferred embodiment, before and/or during step a), the heterogeneous material is crushed, cut, grinded, thin-sectioned and/or polished to obtain the flat surface of the sample.

However, such treatments of the heterogeneous material are optional and can be omitted. Especially the heterogeneous material provided in step a) can be an untreated material, especially a material that has not been crushed, cut, grinded, thin sectioned and/or polished.

In another particularly preferred embodiment, the sample, especially the flat surface of the sample, is subjected to a surface treatment to increase a contrast between the continuous phase and the constituents, especially the particle-shaped constituents. For example, the surface treatment is selected from coloring with an ink and/or polishing with a powder and/or paste.

In particular, a reference scale can be placed on and/or next to the sample.

A reference scale might for example be a character, a geometrical form, a ruler and/or a reference object of known size in the sample area. This allows for a precise determination of the size of the predefined sample area and a precise extraction of the one or more properties in step c).

Preferably, the computer device, especially the mobile computer device, is configured for automatically determining the size of the sample based on the reference scale.

Especially, the sample is placed on a predefined sample area, which in all directions of space is larger than the sample, and preferably, the sample area comprises a reference scale and/or has a known size.

The predefined sample area can be a two-dimensional sample area or a three-dimensional sample area.

The two-dimensional sample area preferably is a flat area, especially a flat rectangular area. In particular, the two-dimensional sample area is aligned horizontally and/or it is a horizontal sample area.

Preferably, the predefined sample area, especially the two-dimensional sample area, comprises a reference scale and/or has a known size.

Preferably, the computer device, especially the mobile computer device, is configured for automatically determining the size of the predefined sample area, especially the two-dimensional sample area, based on the reference scale.

In addition or alternatively, the predefined sample area, especially the two-dimensional sample area, has a predefined known size. In this case, preferably, the computer device, especially the mobile computer device, is configured for setting a predefined size, e.g. from a list of predefined sizes. In this case, no reference scale is required.

Especially, a thin sheet material, preferably of predefined size, is used as the predefined sample area, especially the two-dimensional sample area. For example a sheet of paper, e.g. a DIN A5, A4, or A3 paper, is used as the thin sheet material. Also, papers with other formats, such as e.g. tabloid, letter or statement format, can be used. Additionally, a reference scale can be present on the thin sheet material. Preferably, the computer device, especially the mobile computer device, is configured for automatically determining the size of the predefined sample area, especially the two-dimensional sample area.

Papers as predefined sample areas are readily available and rather cheap.

In general, the size of the predefined sample area, especially the two-dimensional sample area, affects the minimum identifiable size of the constituents, especially the particle-shaped constituents. The smaller the size of the sample area, the smaller constituents, especially particle-shaped constituents, can be identified in the predefined sample area, especially the two-dimensional sample area. Thus, for characterizing small constituents, especially small particle-shaped constituents, small predefined sample areas, especially small two-dimensional sample areas, are beneficial. Small constituents in particular are constituents with a maximum Feret diameter measured along a direction of maximum extension of the constituent not higher than 0.5 mm, preferably not higher than 0.1 mm, especially no higher than 0.063 mm. Small particle-shaped constituents in particular are constituents with a particle size D90 not higher than 0.5 mm, preferably not higher than 0.1 mm, especially no higher than 0.063 mm.

Particle sizes for example can be determined by laser light diffraction as described in ISO 13320:2009.

Especially, the predefined sample area, in particular the thin sheet material, has a specific color different from the color of the constituents, especially the particle-shaped constituents, and/or the continuous phase.

Especially, the specific color of the predefined sample area, especially the two-dimensional sample area, is black. This results in a high contrast when characterizing heterogeneous materials typically used as hardenable compositions, such as e.g. mortar or concrete compositions. However, for other heterogeneous materials, different specific colors of the predefined sample area might be preferable.

Preferably, the sample area is chosen such that the sample is fully surrounded by a frame of a different color. This helps to identify the sample in the digital image.

According to a further preferred embodiment, a light emitting luminous surface is used as the predefined sample area, especially the two-dimensional sample area. Thereby, the luminous surface in particular is illuminated with a light source such that the luminous surface emits light with a homogeneous light distribution over the whole surface.

This is in particular advantageous if the sample of the heterogeneous material is provided in the form of a partly transparent sample, e.g. a thin-ground section or a foam, that can be transilluminated by the light emitting luminous surface.

Especially, a light table pad, in particular a light pad, is used as the predefined sample area. This is an advantageous possibility to provide a light emitting luminous surface. A light table comprises a flat and luminous surface to be oriented horizontally that is illuminated with a light source from the backside.

Especially, the luminous surface consists of a translucent layer that is illuminated from the backside with the light source.

A light pad in particular is a thin light table with a thickness of less than 20% or less than 10% of the width and less than 20% or less than 10% of the length of the luminous surface. Light tables and light pads are known, e.g. in the field of graphics, and commercially available.

When using a light emitting luminous surface, especially a light table, as the sample area, the sample is arranged on top of the luminous surface, especially on the frontside of the luminous surface.

Compared to other predefined sample areas, such as e.g. papers, luminous surfaces, especially light tables or light pads, are in particular beneficial since shadows of the sample can be omitted, the contrast can be increased, less artefacts are produced, better constituent recognition, especially particle recognition, especially of bright constituents, especially bright particles, and/or small constituents, especially small particles, is possible, and the fit of calculated outlines can be improved which in turn increases accuracy of shape parameter, especially particle shape parameter, and size determination, especially particle size determination. Overall, the accuracy of the results of step c) can be improved. Especially, the luminous surface is illuminated such that it emits a color different than the color of the sample, preferably the luminous surface is illuminated such that it emits white light.

In particular, the light source comprises a source of withe light. Optionally the light source additionally comprises a source of light of a color different from white. In particular, the color of the light source is switchable between the different colors. Colors different than white enhance detection of whitish, bright samples.

In particular, the light source is an LED light source. Compared to other light sources, this allows for minimizing heat evolution on the translucent surface or in the sample area, respectively. This reduces the risk of thermally induced changes of the sample.

LED may cause temporal light modulation disturbances. Temporal light modulation is a change in the luminous quantity or spectral distribution of light over time. Such modulations may result in undesirable visual perceptions such as flickering, stroboscopic effects and phantom array effects. Such effects are also known as temporal light artefacts. Such effects are described for example by J.A. Veitch et al in “On the state of knowledge concerning the effects of temporal light modulation” Lighting Res. Technol. 2021, 53, 89-92. It is preferable within the present context, to avoid such temporal light modulation and effects resulting therefrom. Thus, according to some embodiments, the light source is configured to avoid effects resulting from temporal light modulation.

Preferably, the light emitting luminous surface, especially a light table or a light pad, is configured such that the light intensity of the luminous surface can be adjusted, especially continuously or in discrete steps. For example, the light emitting luminous surface, especially the light table or the light pad, is configured such that the light intensity can be switched between 2-5, especially 3-4, different light intensities.

In a further preferred embodiment, the light emitting luminous surface, especially the light table or the light pad, is configured such that it emits polarized light. This can e.g. be achieved by using a light source producing polarized light and/or a polarization filter arranged behind, on top of and/or within the luminous surface. A polarization filter can e.g. be selected from a foil. Polarized light can be used to further enhance the determination of the one or more properties in step c). A foil can also be a colored foil.

If the light emitting luminous surface, especially the light table or the light pad, is configured such that it emits polarized light, there is preferably an analyzer for the polarized light, in particular a polarizer, placed between the sample and the camera. Especially, an analyzer in the form of a foil and/or a filter is used. The analyzer can e.g. be mounted on the camera and/or be placed in between the camera and the sample. Using an analyzer allows for example for selectively enhancing light emission from specific parts of the sample and/or reducing light emission from other parts of the sample. Thereby, for example, a contrast in the image can be improved, certain constituents of the sample can be made visible in the image.

In further preferred embodiments, the emitted light is such that it does not cause interferences.

Furthermore, the luminous surface, especially of a light table or a light pad can, be covered with a protective foil, e.g. for increasing scratch resistance, whereby preferably the protective foil is transparent with respect to the emitted light of the luminous surface. In particular, the protective foil is made of synthetic material. Especially, the protective foil is a replaceable foil.

Particularly, the light emitting luminous surface, especially the light table or the light pad, comprises a frame surrounding the light emitting luminous surface, whereby, the frame has a color different than the light emitting luminous surface, especially a darker color than the light emitting luminous surface, in particular a black color. This helps to identify the predefined sample area, especially the two-dimensional sample area, in the digital image.

For example, as size of the light emitting luminous surface, especially of the light table or the light pad, is equal to the size of a DIN A5, A4, or A3 paper or has the size of the tabloid, letter or statement format. Typically, light pads are slightly bigger in size than any of the afore mentioned formats to ensure that a paper of a given format would fit perfectly to a light pad of such format. Thus, the actual size of the light emitting luminous surface, especially of the light table or the light pad, may also be slightly bigger than any of the afore mentioned formats. Preferably, the computer device, especially the mobile computer device, is configured for manually and/or automatically determining the size of the light emitting luminous surface.

Taking the at least one digital image of the sample with the camera preferably is performed under daylight conditions and/or with a light source, e.g. a flashlight, for illuminating the sample. Thereby, the light used for illuminating the sample preferably is well dispersed in order to avoid shadows and light inhomogeneities. The computer device, especially the mobile computer device, preferably is configured for automatically adjusting light conditions in order to obtain a balanced exposure.

In a further preferred embodiment, the sample or parts thereof can be treated with a luminescent dye, e.g. a fluorescent dye and/or a phosphorescent dye. Thereby, excitation of the dye can e.g. be effected with a light pad and/or a light source directed to the sample. A luminescent dye can be used to further improve the image quality.

In a further preferred embodiment, the camera is aligned plane-parallel, especially horizontally, to the sample and/or the predefined sample area, especially the two-dimensional sample area. Especially preferred, the camera is aligned plane-parallel to a flat surface of the sample, whereby preferably the flat surface is oriented horizontally.

Especially, the camera is aligned plane-parallel if its optical axis runs perpendicular to the sample, the predefined sample area and/or the flat surface of the sample. The optical axis is an imaginary line that defines the path along which light propagates through the camera system

Preferably, the computer device, especially the mobile computer device, is configured for automatically warning the user and/or for preventing taking the at least one image as long as there is a non-plane-parallel alignment. In this case, the computer device, especially the mobile computer device, and/or the camera preferably comprises at least one position sensor which can be assessed when performing the inventive method.

Especially, the camera is aligned horizontally, in particular horizontally and plane-parallel to the sample and/or the predefined sample area, when taking the at least one digital image of the sample in step b). Thereby, preferably, the sample and/or the predefined sample area is aligned horizontally.

Especially, the camera is aligned horizontally if its optical axis runs vertically.

Taking the at least one digital image of the sample in step b) with the camera in horizontal alignment, in particular plane-parallel to the sample, greatly simplifies the image capturing process. Specifically, by adjusting the height of the camera over the area, a share of the sample in the image can be easily maximized with the horizontal alignment.

Also, a horizontal alignment of the sample and/or the predefined sample area is beneficial when analyzing fluid samples, such as e.g. emulsions, since the fluid will automatically remain stable and motionless in its position during the image capturing process. Thereby, the predefined sample area can for example be located on a table or any other essentially horizontal surface.

However, the method can be implemented as well with a non-plane-parallel alignment of the camera.

In a further preferred embodiment, at least two consecutive digital images of the sample are taken. In this case, preferably, step c) is performed with the at least two digital images. This can be helpful for increasing the number of statistical counts and for more precisely determining the one or more properties in step c). In particular, in step c), the at least two digital images are superimposed.

Especially, when taking the at least one image, the camera is aligned so that a share of the sample and/or the sample area in the image is maximized. This can be implemented for example by providing alignment instructions to the user and/or by automatically adjusting at least one setting of the camera, e.g. the focal length of the camera. Thus, preferably, the computer device, especially the mobile computer device, is configured accordingly.

Highly preferred, a minimum detectable size, especially a minimum detectable particle size, of the constituents, especially the particle-shaped constituents, is calculated, preferably by taking into account the resolution of the camera, the area share of the sample and/or the sample area in the total area of the image, and the real size of the sample area. Preferably, the computer device, especially the mobile computer device, is configured accordingly.

Especially, if the minimum detectable size is below a predetermined threshold, a warning is provided to the user, alignment instructions are provided to the user and/or a setting of the camera, e.g. the focal distance, is adjusted. The predetermined threshold can e.g. be set manually. This helps to avoid taking images under unsuitable conditions.

The image analysis of the at least one digital image in step c) can be implemented with known and readily available image analysis algorithms, e.g. by using software packages and/or libraries provided in Matlab (by MathWorks®), OpenCV (cf. https://opencv.org), ImageJ (by Wayne Rasband; cf. https://imagej.net) and/or artificial intelligence algorithms.

Especially, in step c) at least one particle size parameter is extracted. Thereby, preferably, it comprises at least one of the following parameters:
the particle size distribution of the population of particles identified in the at least one digital image; and/or
at least one statistical parameter selected from the group of average particle size, mean diameter, and/or Dₓ-value with x=0-100, especially D₁₀, D₅₀, D₈₅ and D₁₀₀ values; and/or
a deviation from a predefined nominal value and/or nominal distribution, e.g. from fuller curves, standard sieving curves for specific concrete types; and/or
the fineness modulus.

According to especially preferred embodiments, the at least one particle size parameter extracted comprises or is the particle size distribution of the population of particle-shaped constituents identified in the at least one digital image.

These are highly relevant parameters when providing formulations of hardenable compositions with particle-shaped constituents.

Thereby, the extracted particle size distribution in particular is meant to correspond to the particle size distribution as defined in standard EN 933-1:2012.

Another possible way to determine particle size distribution is laser light diffraction as described in ISO 13320:2009. Thus, the extracted particle size distribution in especially is meant to correspond to the particle size distribution as defined in standard ISO 13320:2009.

However, depending on the sample and the information required, the particle size distribution can be defined differently.

A Dx value has the meaning that a proportion of the given assemblage of particles of x % has a lower particle size than the given value. Thus, a D₉₀ value, for example, means that 90% of the assemblage of particles has a particle size smaller than the D₉₀ value given. Accordingly, the average particle size, in particular the median particle size, corresponds in particular to the D₅₀ value (50% of the particles are smaller than the given value, 50% are correspondingly bigger). Especially, the percent (%) are volume-%.

In general, the at least one particle size parameter extracted preferably comprises at least the particle size distribution of the population of particle-shaped constituents identified in the at least one digital image.

Especially, for particle sizes below the minimum detectable particle size, the particle size distribution may be extrapolated based on the extracted particle size distribution and/or it may be equated with a predefined standard distribution. For example, polynomial extrapolation is used, especially based on Lagrange interpolation or using Newton's method of finite differences to create a Newton series that fits the extracted particle size distribution.

Especially, in step c), at least one particle shape parameter is extracted. Preferably it comprises at least one of the following parameters:
roundness,
sphericity,
aspect ratio,
roughness,
solidity,
flakiness index,
shape index,
percentage of crushed and broken surfaces, and/or
angularity;
distances and/or angles between surface structures of individual particles.

These are parameters that have significant influence on the workability of hardenable compositions. See for example “Correlation between Shape of Aggregate and Mechanical Properties of Asphalt Concrete”: Digital Image Processing Approach: Road Materials and Pavement Design: Vol 12, No 2.

Especially, the particle shape parameters are meant to correspond to the shape parameters as defined in standards EN 933-1:2012 to -7:2012.

Especially, the particle shape parameters include the aspect ratio expressed as the maximum Feret diameter to the minimum Feret diameter.

Further shape parameters which can be extracted are given in Blott and Pye, Sedimentology (2008) 55, 31-63.

In particular, the at least one particle shape parameter is extracted with regard to particle-shaped constituents of a predetermined size only, especially to particle-shaped constituents larger than a given threshold size. For relatively small particle-shaped constituents, the particle shape of particle-shaped constituents affects the properties of hardenable or hardened compositions less. For example, the at least one particle shape parameter is extracted with regard to particle-shaped constituents>0.5 mm, in particular>1 mm.

Nevertheless, it is possible to analyze the shape parameters of all particle-shaped constituents if desired.

Furthermore, it is possible to extract the at least one particle shape parameter with regard to particle-shaped constituents larger than a given lower threshold size and particle-shaped constituents smaller than a given upper threshold size. Thereby, in particular, at least two, at least three or more different particle shape parameters can be extracted simultaneously within the range between the lower threshold and the upper threshold.

This allows for identifying shape parameters of particle-shaped constituents with specific sizes, which are for example known to affect certain properties of interest of hardenable compositions, such as e.g. the rheology of a hardenable compositions in processable state.

According to another preferred implementation, for each of at least two or more predefined size fractions, e.g. sieve size fractions, of the particle-shaped constituents, at least one individual particle shape parameter is extracted.

Thereby, especially, at least one individual mean value of the at least one particle shape parameter is extracted for each size fraction. This allows for providing detailed information about the size dependent distribution of the particle shape parameters.

For example, a mean value of the at least one particle shape parameter is provided for each of the least two or more predefined particle size fractions, e.g. sieve size fractions, separately. Optionally, the particle counts per size fraction can be normalized by the volume of the particle-shaped constituents considered, e.g. in order to obtain data comparable to particle size distributions. In addition, in this case, at least two, at least three or more different particle shape parameters can be provided simultaneously.

Such data can e.g. be presented in a bar plot with the predefined particle size fractions as categories and the corresponding mean values of the at least one particle shape parameters in the form of bars with heights or lengths proportional to the values that they represent.

Furthermore, it is possible to provide the mean value of the at least one particle shape parameter with regard to particle-shaped constituents in several selected size fractions, e.g. particle-shaped constituents being present in size fractions above and/or below a given fraction threshold. Similar to the above, this allows for identifying shape parameters of particles in size fractions, which are for example known to affect certain properties of interest of hardenable or hardened compositions.

For determining the spatial distribution and/or the segregation parameter, which might be extracted in step c), the local densities of the constituents, especially the particle-shaped constituents, can be determined for at least two or more sub-areas of the sample. If the local densities are identical in all sub-areas, the spatial distribution is homogeneous. Otherwise, there is an inhomogeneous distribution, e.g. caused by segregation of the constituents, especially the particle-shaped constituents.

In a special embodiment, the constituents, especially the particle-shaped constituents, have an elongate shape. In this case, an orientation parameter, especially a particle orientation parameter, can be extracted in step c). In particular, the constituent orientation parameter is a constituent angle distribution, especially a particle angle distribution, and/or an average constituent angle, especially an average particle angle distribution. The angle can e.g. be defined as the angle between the direction of a longitudinal axis of the elongate constituents, especially particle-shaped constituents, and a predefined reference direction, e.g. an edge of the at least one digital image. An average constituent angle, especially an average particle angle, can be defined as a mean value of all constituent angles, especially particle angles, e.g. the arithmetic mean value. For example, if the arithmetic mean value of all constituent angles, especially particle angles, is different from zero, the elongate constituents, especially particle-shaped constituents, have a predominant orientation in the heterogeneous material.

In a further preferred implementation, for each of the at least one digital image, an outline image is generated. An outline image is also called a contour image and comprises the outlines of all of the identified constituents, especially particle-shaped constituents, in the digital image. The outline image can be made available in step d) via a user interface, via a machine interface and/or on a data storage medium. For example, the outline image can be displayed within the application and/or stored on an external server. The outline image can serve as tool to evaluate the quality of the digital image and/or the analysis performed.

Especially, in step b), at least two, preferably at least three, in particular at least five or at least ten, digital images are taken and for each image an image analysis is performed in step c), and by taking into account each of the at least one properties individually extracted from the at least two images, a deviation, especially the standard deviation, of the at least one property is determined. The deviation can serve as tool to evaluate the quality of the digital images and/or the analysis performed.

Especially, if a deviation is above a predetermined threshold, a warning can be provided to the user and/or if a deviation is above a predetermined threshold, a digital image and/or an outline image giving rise to diverging parameters might be identified and/or indicated.

Preferably, the inventive method further comprises the step of assigning at least one attribute of the sample. Especially, the attribute is selected from:
a unique identifier of the sample;
a chemical and/or physical property of the sample, especially
   the type of the sample, e.g. mortar, concrete, grout, screed, flooring adhesive, or coating compositions;
   raw materials used for preparing sample;
   mix design, especially proportions of individual raw materials, of the sample, e.g. proportions of binders, aggregates, additives, water, etc.;
   density of sample;
   application method, e.g. casting, spraying, additive manufacturing, etc.;
   special treatments received during production, e.g. mixing, compaction, vibration, heating, etc.;
   processing properties of sample during production, e.g. rheological properties, e.g. flow properties, slump flow, viscosity, t50 time, yield stress and/or consistency class, of sample in processable state, especially in case of hardenable samples such as mortars, concretes, grouts, etc.;
   curing properties, e.g. setting and/or hardening times, in case of hardenable or hardened sample;
   mechanical properties of the sample, e.g. strength class, compressive strength, flexural strength, hardness;
   durability, e.g. exposition class, chemical resistance, freeze-thaw resistance, etc.;
   a maximum grain size of the particle-shaped constituents;
   a minimum grain size of the particle-shaped constituents;
   the state of the sample, e.g. fluid or hardened; and/or
   pretreatments received, e.g. crushed, cut, grinded thin-sectioned and/or polished.
descriptive information of the sample, especially
   the location of the source of the sample, e.g. manually or automatically by position sensors;
   the intended use, e.g. a project name and/or a customer name; and/or
   a general comment.

Preferably, the computer device, especially the mobile computer device, is configured to query the at least one attribute of the sample attributes, especially before, during and/or after steps a) to b).

Especially, a unique identifier of the sample is generated automatically.

In particular, the inventive method is performed to obtain one or more of the following characteristics of the heterogeneous material, especially a hardened binder composition:
The particle size distribution of aggregates in the heterogeneous material;
The particle shape of aggregates in the heterogeneous material;
The quantity of a binder share, especially a paste share, in the heterogeneous material;
The quality of recycled aggregates and/or other raw materials, especially measured by determining the quantity and/or share of binder present on recycled aggregates;
A mix ratio of the heterogeneous material with respect to the particle-shaped constituents and the continuous phase;
A ratio of coarse aggregates to cement (ca/c), e.g. in mortar or concrete materials;
The distribution of aggregates in the heterogeneous material;
The share, size and/or distribution of air voids in the heterogeneous material;
The quantity and/or quality of the mineral composition in the heterogeneous material, whereby the quality is in particular measured by determining the share of one or more particle shaped mineral constituents;
The quality of the production method and/or special treatments received during production, for example the quality of compaction (e.g. vibration) and/or the quality of the application method, whereby the quality is in particular measured by determining the homogeneity of the sample and/or the distribution of particle shaped constituents;
The color distribution at the surface of the heterogeneous material, e.g. for characterizing carbonization and/or efflorescence, in particular for determining ageing of the sample;
The determination of a failure mode of the heterogeneous material, especially by measuring the number, size, shape and/or orientation of cracks in the sample;
Monitoring of mechanical defects in the heterogeneous material, e.g. cracks;
An area share of cracks with respect to the area of the continuous phase, especially in a synthetic material, in particular in a synthetic membrane;
A size, shape, spatial distribution, orientation, packing density, and/or segregation of cracks, especially in a synthetic material, in particular in a synthetic membrane;
Workability related information, especially rheological properties, e.g. flow properties, slump flow, viscosity, t50 time, yield stress and/or consistency class; e.g. by taking one or more pictures of the sample in processable state, e.g. of the slump flow of the sample, at a predefined time or at predefined time intervals;
Predictive modelling, in particular the prediction of adjustments for producing further samples, e.g. adjustment of raw material(s) and/or mix design adjustments, especially for improving certain properties of the further sample.

In particular, the method is at least partly, especially completely, performed on the computer device, especially the mobile computer device.

In case the method is completely performed on the computer device, especially the mobile computer device, the complete characterization can take place on the computer device, especially the mobile computer device, without requiring any communication network. Also, the at least one digital image, preferably together with the at least one attribute can be stored on the computer device, especially the mobile computer device, e.g. for sharing, recalling, further evaluation, and/or predictive modelling.

Preferably, the computer device, especially the mobile computer device, is configured such that the at least one digital image, preferably together with the at least one attribute can be transferred to an external device via communication means, e.g. a communication interface of the computer device, especially the mobile device, for storing. Likewise, the computer device, especially the mobile computer device, preferably is configured for recalling the stored data from the external device. According to another preferred implementation, the image analysis in step c) and/or the making available in step d) is/are conducted on a separate computer device, e.g. on a server.

In this case, preferably, the at least one digital image, preferably together with the at least one attribute is transferred to the external device via communication means, e.g. a communication interface of the mobile device. Such a decentralized solution is beneficial since computationally intensive step c) can be performed on another device which in turn helps to increase runtime of the computer device, especially the mobile computer device. Furthermore, steps c) and/or d) can be optimized by updating the software part on the side of the external device without the user having to update the software part on the computer device, especially the mobile computer device.

Thereby, if there is no communication network available, the at least one digital image, preferably together with the at least one attribute, can be stored temporarily on the computer device, especially the mobile computer device, and transferred to the external device later on when the communication network is available.

In this case, the image, preferably together with the at least one attribute, is stored on an external computer device, e.g. a server, in particular for sharing, recalling and/or further evaluation.

In step d) the one or more of the properties extracted are made available via a user interface, via a machine interface and/or on a data storage medium. If desired, the at least one digital image, optionally together with the at least one attribute, can be made available in addition. However, this is not a requirement since for daily work the image as such is hardly important for a user.

If the data is made available via a user interface, this can be done directly on the computer device, especially the mobile computer device, and/or an external computer device. Thereby, for example, the one or more of the properties extracted, e.g. the at least one particle size parameter and the at least one particle shape parameter, are plotted in a graph, e.g. the particle size distribution, and/or presented in a dynamic plot, e.g. in a roundness versus sphericity plot or the bar plot of mean particle size parameter versus sieve opening.

Making available the data via a machine interface allows for transferring the data to an external computer device, e.g. a server and/or a desktop computer.

Also, it is possible to make available the data on a data storage medium, e.g. on an internal data storage medium of the computer device, especially the mobile computer device, a storage device attached to the computer device, especially the mobile computer device, and/or on a storage device of an external computer.

Especially, the one or more of the properties extracted, optionally together with the at least one attribute, and optionally with the at least one image, are written in a data file with predefined file format. E.g. the file format is chosen from json, csv, txt and/or pdf. However, other file formats can be used as well. Typically, the data file does not include the at least one image. This helps to reduce the file size.

In particular, the data file is transferred to another application, a further computer device, especially a further mobile computer device, and/or an external computer device. Transfer can be performed by any kind of communication means, e.g. wireless communication and/or wired communication. This allows a user to share the characterization of the constituents, especially solid particles, with other users, transfer it to another application for further evaluation and/or to a data storage server. Thus, preferably, the computer device, especially the mobile computer device, is configured for transferring a data file to another application, a further computer device, especially a further mobile computer device, and/or an external computer device.

Especially, the distributed constituents, especially particle-shaped constituents, are optically distinguishable from the continuous phase.

Especially, the distributed constituents, especially the distributed particle-shaped constituents, have different light absorption and/or light reflection properties than the continuous phase, in particular with respect to light having a wavelength in the range of 200nm-5′000 nm, especially in the range of 380-780 nm.

A size of the constituents, especially particle-shaped constituents, for example ranges from >0 to 125 mm, preferably from 10 μm to 32 mm, more preferably from 0.063 mm to 16 mm, especially from 0.1 mm to 2 mm. Such kind of constituents, especially particle-shaped constituents, are typically present in hardenable compositions such as e.g. mortar or concrete compositions. However, the method can be used for characterization particle-shaped constituents with other sizes as well.

In particular, the heterogeneous material is a hardenable or hardened binder material, especially a hardenable or hardened mineral binder composition or hardenable or hardened organic binder composition.

The hardenable or hardened binder material can be present in fluid state, e.g. during processing and/or the hardening process. Also, the hardenable or hardened binder material can be in the solid state, e.g. after partly hardening or after completion of the hardening process.

A hardenable or hardened binder material is for example selected from mortars, concretes, grouts, screeds, floorings, adhesives, or coatings. Thereby, the binder can be a mineral binder, an organic binder or a hybrid binder comprising mineral binder and organic binder in combination.

An “organic binder” in particular a polymeric resin. For example the organic binder is a resin based on epoxide, polyurethane, acrylate, polyester and/or polychloroprene.

The expression “mineral binder” means in particular a binder which in the presence of water reacts in a hydration reaction to give solid hydrates or hydrate phases. This can by way of example be a hydraulic binder (e.g. cement or hydraulic lime), a latently hydraulic binder (e.g. slag), a pozzolanic binder (e.g. fly ash), or a non-hydraulic binder (e.g. gypsum or white lime).

The entire mineral binder advantageously comprises a proportion of at least 5% by weight of the hydraulic binder, in particular at least 20% by weight, preferably at least 50% by weight, specifically at least 75% by weight. In another advantageous embodiment the mineral binder comprises at least 95% by weight of hydraulic binder, in particular cement.

The mineral binder in particular comprises a hydraulic binder, preferably cement. Particular preference is given to Portland cement, in particular of the type CEM I, II, III, or IV (in accordance with the standard EN 197-1). However, it can also be advantageous that the binder composition comprises, in addition or instead of a hydraulic binder, other binders. These are in particular latently hydraulic binders and/or pozzolanic binders. Examples of suitable latently hydraulic binders and/or pozzolanic binders are slag, fly ash, and/or silica dust. In one advantageous embodiment the mineral binder comprises from 5 to 95% by weight, in particular from 20 to 50% by weight, of latently hydraulic binders and/or pozzolanic binders.

In a special embodiment, the mineral binder comprises a mixture of calcined clay, limestone, and Portland cement.

The term “clay” refers to a solid material composed to at least 30 wt.-%, preferably to at least 35 wt.-%, especially to at least 75 wt.-%, each relative to its dry weight, of clay minerals. A calcined clay is a clay material that has been put to a heat treatment, preferably at a temperature between 500-900° C., or in a flash calcination process at temperatures between 800-1100° C. According to especially preferred embodiments of the present invention, the calcined clay is metakaolin.

In preferred embodiments of the present invention the chemical compositions of limestone and Portland cement are as defined in standard EN 197-1:2011. In the alternative, limestone may also stand for magnesium carbonate, dolomite, and or mixtures of magnesium carbonate, dolomite, and/or calcium carbonate. It is especially preferred that limestone within the present context is a naturally occurring limestone mainly consisting of calcium carbonate (typically calcite and/or aragonite) but typically also containing some magnesium carbonate and/or dolomite. Limestone may also be a naturally occurring marl.

According to preferred embodiments, Portland cement is of type CEM I. According to embodiments, the Portland clinker content in a Portland cement of the present invention is at least 35 w %, preferably at least 65 wt.-%, especially at least 95 wt. %, each based on the total dry weight of the cement.

According to embodiments, the mineral binder comprises calcined clay, limestone, and Portland cement in the following weight ratios:
P:CC is from 33:1 to 1:1, preferably from 8:1 to 1:1,
CC:L is from 10:1 to 1:50, preferably from 10:1 to 1:33, more preferably from 5:1 to 1:10, and
P:L is from 20:1 to 1:4, preferably from 5:1 to 1:1.

According to embodiments, the mineral binder consists to at least 65 wt.-%, preferably at least 80 wt.-%, more preferably at least 92 wt.-%, in each case relative to the total dry weight of the mineral binder, of calcined clay, limestone, and Portland cement.

According to embodiments of the present invention, a mineral binder comprises a mixture of
a) 25-100 mass parts of Portland cement (P),
b) 3-50 mass parts of calcined clay (CC), especially of metakaolin,
c) 5-100 mass parts of limestone (L).

According to another preferred embodiment, the heterogeneous material is a synthetic material, especially a synthetic membrane, in particular made from polyvinyl chloride (PVC), and/or thermoplastic polyolefin (TPO), e.g. from the group comprising high-density polyethylene (HDPE), medium-density polyethylene (MDPE), low-density polyethylene (LDPE), polyethylene (PE), polyethylene terephthalate (PET), polystyrene (PS), polyvinyl chloride (PVC), polyamides (PA), ethylene/vinyl acetate copolymer (EVA), chlorosulfonated polyethylene, thermoplastic polyolefin elastomer (TPO, TPE-O), ethylene propylene diene rubber (EPDM), and mixtures thereof.

Such a membrane can e.g. be a waterproofing membrane or a roofing membrane.

Under long term outside exposure of polymeric materials, such as roofing membranes, surface cracks will appear and eventually lead to failure of the product. When inspecting a condition of a roof, it is thus a standard procedure to check the condition of the membrane by evaluating the crack intensity on the surface of the membranes.

In this case, the constituents may for example be cracks, especially as described in the following. Thanks to the inventive method, the cracks can be analyzed in a direct and efficient manner. This allows for efficiently analyzing the condition of a membrane. Crack analysis in membranes can e.g. be done in line with standard EN 13956:2013.

In particular, the particle-shaped constituents of the sample are solid particles, e.g. selected from sand, aggregates, natural or synthetic fibers, glass spheres, sand replacements, manufactured sands, crushed and/or recycled construction materials, bio aggregates, metallic particles, ashes, mine tailings and/or polymeric particles. However, other particle-shaped constituents can be present as well.

In a special embodiment, the constituents, especially particle-shaped constituents, of the sample are discolored particle-shaped spots and/or flakes of the sample.

In another embodiment, the distributed constituents, especially distributed particle-shaped constituents are gas-filled pores, especially bugholes. Bugholes are surface air voids present in hardened binder compositions, such as e.g. adhesive, coatings, grouts, mortar or concrete compositions.

According to a further embodiment, the distributed constituents are cracks, especially unbranched and/or branched cracks, whereby the cracks in particular are gas-filled. Such kind of cracks can for example be partially or completely straight cracks and/or partially curved or fully curved cracks.

In a special embodiment, the cracks are cracks in a synthetic material, especially a synthetic membrane as described above.

In particular, the heterogeneous material comprises more than one type of distributed constituents, especially particle-shaped constituents, which are distinguishable in the at least one digital image, e.g. because of different colors, the extraction for one or more of the properties in step b) is performed for each type of distinguishable constituents, especially particle-shaped constituents, separately.

Thereby it is for example possible to analyze the sample with respect to different properties at the same time. For example, it is possible to analyze in a binder composition the distribution of air voids and the distribution of aggregates simultaneously.

The continuous phase can be a solid or a liquid phase. A solid continuous phase is for example a hardened binder composition, e.g. comprising a mineral and/or an organic binder. A liquid continuous phase is for example a solvent, e.g. water, alcohol, or a fluid binder, e.g. cement mixed with water, before hardening is completed.

Especially, the continuous phase has a different appearance, in particular a different color, than the distributed constituents, especially particle-shaped constituents, and/or there is an interface between the distributed constituents, especially particle-shaped constituents, and the continuous phase, which can be detected in the at least one digital image.

In particular, the heterogeneous material to be characterized is a solid material.

However, in another preferred embodiment, the heterogeneous material is a fluid material, especially a liquid material. A heterogeneous material in the form of a fluid is for example selected from an emulsion, a foam, a suspension, a workable binder composition. However, other heterogeneous materials in fluid form can be used as well.

In another preferred embodiment, the distributed constituents, especially the distributed particle-shaped constituents, comprise a first type of mineral material and the continuous phase of condensed matter comprises as second type of a mineral material, which is different from the first type of mineral material. Such a situation is for example given, if the inventive method is used for analyzing heterogeneous mineral materials.

A still further aspect is directed to a system comprising a computer device, especially a mobile computer device, and optionally an additional separate computer device, whereby the system comprises:
(i) means for carrying out steps a) to d), of the method as described above, and/or
(ii) means for carrying out at least the steps a) and b), especially steps a) and b) and d), of the method as described above, and means for transferring at least one digital image of the sample, optionally together with at least one attribute, to the separate computer device.

Another aspect of the invention is related to a system comprising a computer device, whereby the system comprises means for receiving at least one digital image of a sample of a heterogeneous material comprising distributed constituents, especially particle-shaped constituents, dispersed within a continuous phase of condensed matter and means for carrying out at least steps c) and/or d) of the method as described above.

Furthermore, the invention is concerned with a computer-readable medium comprising instructions which, when executed by a computer device, especially a mobile computer device, cause the computer device to carry out at least the steps a) and b), in particular a) and b) and d), especially steps a) to d), of the method as described above.

Also the invention is related to a computer-readable medium comprising instructions which, when executed by a computer device, cause the computer device to receive at least one digital image, optionally together with at least one attribute, and perform at least steps c) and/or d) of the method as described above.

Especially, any data processed and/or produced with the inventive methods is encrypted, especially such that only authorized users can access the original data.

Likewise, any computer program and/or application implementing the inventive methods are encrypted too. Methods for encryption are known to the skilled person.

Further advantageous configurations of the invention are evident from the exemplary embodiments.

### BRIEF DESCRIPTION OF THE DRAWINGS

The drawings used to explain the embodiments show:

FIG. 1 A flow chart of an inventive computer-implemented method;

FIG. 2 A schematic overview of a system comprising means for carrying out the method of FIG. 1;

FIG. 3 A schematic view of the second step of the method of FIG. 1 whereby a user (not shown) holding a smartphone is taking an image of a cuboid hardened mortar sample with sand aggregates;

FIG. 4 A schematic view of the second step of the method of FIG. 1 whereby a user (not shown) holding a smartphone is taking an image of a vertical wall of a building made from concrete comprising aggregates of different sizes;

FIG. 5 an example of the structure of a data file;

FIG. 6 A bar plot of selected particle shape parameters (roundness, sphericity and aspect ratio) per particle sieve size fraction;

FIG. 7 An outline image overlaid over a digital image that was analyzed with the inventive method;

FIG. 8a A photography of the surface of a concrete floor that was analyzed with the inventive method;

FIG. 8b A detailed view of FIG. 8a;

FIG. 9a A photography of the polished surface of a concrete drill core that was analyzed with the inventive method;

FIG. 9b A detailed view of FIG. 9a;

FIG. 10a A photography of a polished surface of granite that was analyzed with the inventive method;

FIG. 10b A detailed view of FIG. 10a;

FIG. 11a A photography of a surface of an epoxy grout that was analyzed with the inventive method;

FIG. 11b A detailed view of FIG. 11a;

FIG. 12a A photography of a surface of a liquid aqueous foam that was analyzed with the inventive method.

FIG. 12b A detailed view of FIG. 12a;

FIG. 13a A photography of a hardened foam that was analyzed with the inventive method;

FIG. 13b A detailed view of FIG. 13a;

FIG. 14a A photography of a concrete wall with bugholes that was analyzed with the inventive method;

FIG. 14b A detailed view of FIG. 14a;

FIG. 15a A photography of a concrete wall with discoloration caused by efflorescence (bright area);

FIG. 15b A detailed view of FIG. 15a;

FIG. 16a A photography taken during a flow table test of a freshly prepared mortar sample;

FIG. 16b A detailed view of FIG. 16a;

FIG. 17a A photography of a mortar sample with air voids that was inverted for determining the share of the bearing area and the air voids with the inventive method;

FIG. 17b A detailed view of FIG. 17a;

FIG. 18 A photography of several recycled aggregates with residues of cement (bright areas) which were analyzed with the inventive method;

FIG. 19a A photography of a synthetic membrane with no cracks (class 0 according to on EN 13956:2013);

FIG. 19b A photography of a synthetic membrane with cracks of class 1 according to on EN 13956:2013;

FIG. 19c A photography of a synthetic membrane with cracks of class 2 according to on EN 13956:2013.

### EXEMPLARY EMBODIMENTS

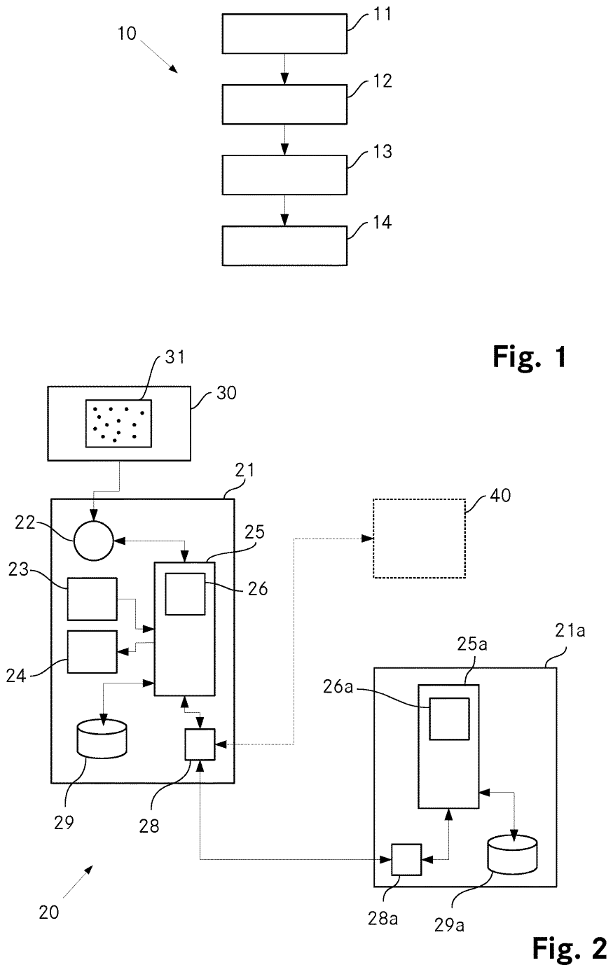

FIG. 1 shows a flow chart of an inventive computer-implemented method 10. In a first step 11, a sample of a heterogeneous material to be analyzed, e.g. a cuboid hardened mortar sample with sand aggregates (particle-shaped constituents) having particle sizes ranging from >0 to 2 mm embedded in a cementitious matrix C (continuous phase of condensed matter), is provided on a two-dimensional sample area of known size. The sample area is for example formed by a sheet of black paper of A4 size.

In a second step 12, a digital image of one of the flat surfaces of the cuboid sample is taken with a camera of a mobile computer device, e.g. a smartphone. The camera for example has a 4K resolution.

Thereafter, in a third step 13, an image analysis of the digital image is performed for extracting at least one particle size parameter, e.g. the particle size distribution, and at least one particle shape parameter, e.g. roundness or sphericity, of the population of the sand particles identified in the at least one digital image. Additionally, the area share of the cementitious matrix C (continuous phase) is determined in the at least one digital image.

In a fourth step 14, the at least one particle size parameter and the at least one particle shape parameter and the area share of the cementitious matrix are made available via a user interface, e.g. a display of the mobile computer device.

FIG. 2 shows a schematic overview of a system 20 comprising means for carrying out the method shown in FIG. 1.

Specifically, the system 20 comprises a smartphone 21 with a camera 22, a touch sensitive display comprising input device 23 and a display 24, a data processing unit 25 with a random access memory, a data storage device 29 and a wireless communication interface 28.

In operation, an application 26 is executed in the data processing unit 25, whereby the application is configured for performing steps 12 and 14 of the method described with FIG. 1.

Specifically, the application 26 assists a user in taking an image of a sample 31 consisting of a heterogeneous material in a two-dimensional sample area 30 with the built-in smartphone camera 22. Thereby, the application is for example configured for automatically warning the user and/or for preventing taking the image as long as there is a non-plane-parallel alignment to a surface of the sample 31. This can be achieved by assessing the position sensors of the smartphone (not shown). Also, the application is configured for automatically adjusting light conditions in order to obtain a balanced exposure. The digital image taken is stored in the random access memory and/or the data storage 29.

Additionally, the application 26 asks the user to enter one or more attributes of the sample via the input device and assigns a unique identifier to the sample. Attributes are e.g. the maximum grain size of the aggregates, the type of the aggregates (natural, crushed, manufactured, recycled; re-used solid particles), the location of the source of the aggregates, the type of cement, the intended use (project name, customer name); and/or a general comment.

The query can e.g. be made by presenting to the user input fields, selection fields, maps and/or text input fields on the display 24 and storing the data provided by the user via the input device 23 together with the at least digital image in the random access memory and/or the data storage 29.

For example, the location of the source of aggregates can be provided manually by the user, e.g. by entering geo coordinates into input fields and/or by marking the position on a map shown on the display 24. It is however possible to automatically provide the location of the source of the aggregates e.g. by sensors of a global navigation satellite system, such as e.g. GPS, Galileo, Beidou and/or Glonass sensors. Thereby, the user might be requested to confirm the automatically determined position.

In the system 20 shown in FIG. 2, step 13 of the method shown in FIG. 1 is performed on an external server 21a. Specifically, the images taken with the smartphone camera 22, optionally together with the attributes, is transferred via the wireless communication interface 28 (or any other communication interface) and a network (e.g. the internet; not shown) to the server 21a. The server 21a receives the data via its communication interface 28a and forwards it to an image analysis application 26a running in a processing unit 25a.

The image, optionally together with the attributes can be stored on a data storage 29a of the server 21a for later sharing, recalling and/or further evaluation.

The application 26a performs an image analysis of the digital image whereby, for example, at least one particle size parameter, e.g. the particle size distribution, and at least one particle shape parameter, e.g. roundness or sphericity, of the population of particles identified in the at least one digital image as well as the area share is extracted. Thereby one or more attributes may be considered in the analysis as well.

The application 26a is implemented for example with image analysis algorithms such as e.g. software packages and/or libraries provided in Matlab, OpenCV and/or ImageJ, and/or with artificial intelligence software.

After the image analysis is finished, the at least one particle size parameter, e.g. the particle size distribution, and at least one particle shape parameter, e.g. roundness or sphericity, as well as the area share are sent back to the smartphone 21 or the application 25 being executed on it, respectively, via the communication interfaces 28a, 28.

The application 25a then makes available the at least one particle size parameter and/or the at least one particle shape parameter and the area share via the display 24, or saves the parameters on the data storage 29, preferably together with the attributes, for later sharing, recalling and/or further evaluation. This data can be stored for example in the form of a data file having a file format chosen from json, csv, txt, pdf, and/or a proprietary file format. Especially, a file format capable of being read by the application called “Sika Mix Design App” and/or any other additional application is chosen. See FIG. 4 for an example.

Additionally, the application 25 is configured for sharing the at least one particle size parameter, the at least one particle shape parameter, the area share and the attributes with another user by sending them, in particular as a data file, via the communication interface 28 to a further computer device 40, e.g. a smartphone of another user. This can for example be initiated by the user via input device 23, e.g. by pressing a button shown on the display 24.

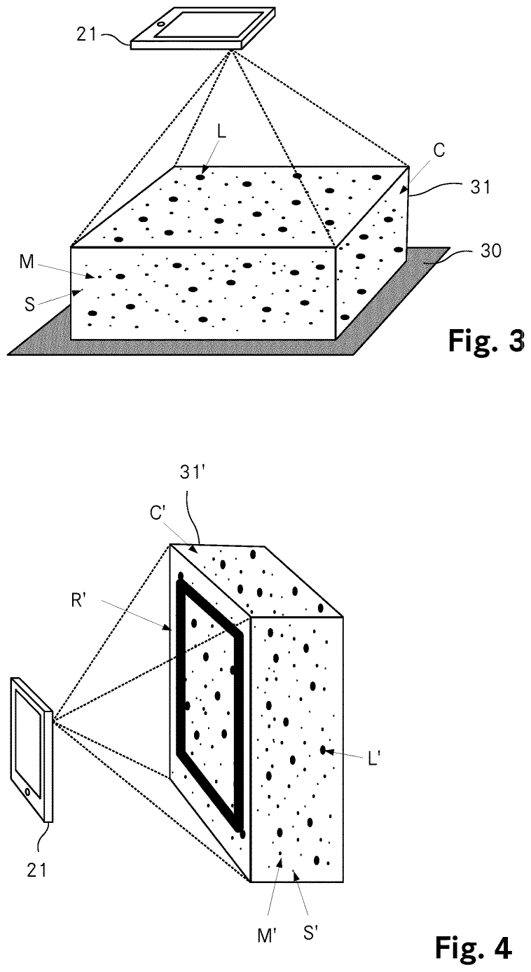

FIG. 3 shows a schematic view of step 12 of the method shown in FIG. 1. Thereby, a user (not shown), holding a smartphone 21 in his hands, takes an image of a sample 31, e.g. a cuboid hardened mortar sample with sand aggregates. Thereby, aggregates of solid sand particles L (large), M (medium), S (small) of different sizes ranging from >0 to 2 mm are embedded within a cementitious matrix C. The sample 31 is provided on a black sheet of paper of A4 size serving as a two-dimensional sample area 30 that in both horizontal directions of space is larger than the sample 31. The smartphone 21 is held in horizontal orientation and plane-parallel to the surface of the sample 31.

FIG. 4 shows another schematic view of step 12 of the method shown in FIG. 1. In this case, the smartphone 21 is held in vertical orientation and plane-parallel to a vertical surface of a sample 31′. The sample 31′ is for example a wall of a building made from concrete comprising aggregates of gravel particles L′ (large), sand particles M′ (medium), and sand particles S′ (small) of different sizes ranging from >0 to 12 mm embedded within a cementitious matrix C′. In this case a black frame-shaped marking R′ has been affixed and/or marked on the sample surface. The marking R′ serves as reference scale that can be used to determine the size of particle shape. If the marking R′ is visible in the picture and the size of marking R′ is set in the smartphone, the real size of particle shape constituents can be determined.

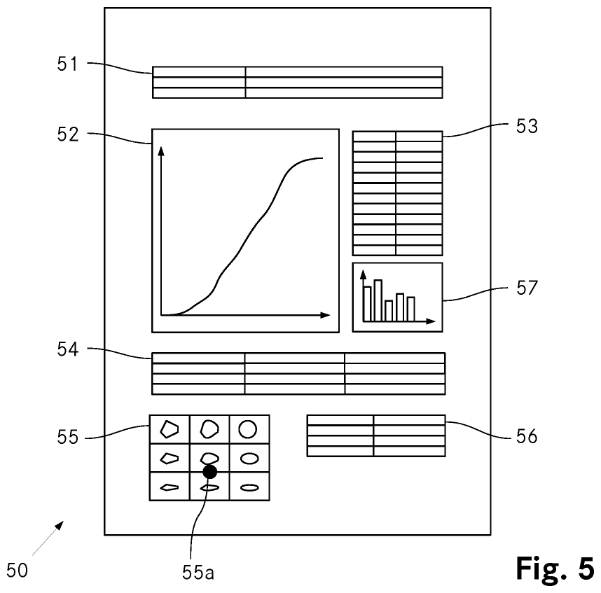

FIG. 5 shows an example of the structure of a data file 50 in pdf file format. The data file 50 comprises a table 51 with attributes provided by the user, e.g. the type of the aggregates (natural, crushed, manufactured, recycled; re-used solid particles), the location of the source of the aggregates, the intended use (project name, customer name); and/or a general comment.

Also, the file 50 comprises a graph 52 representing the particle size distribution, a table 53 comprising the calculated sieve size pass rate of the solid particles and/or the calculated proportion of retained solid particles, and a table 54 with statistical parameters, such as D₁₀, D₅₀, D₈₅, and D₁₀₀ values as well as the fineness modulus of the particle-shaped constituents analyzed.

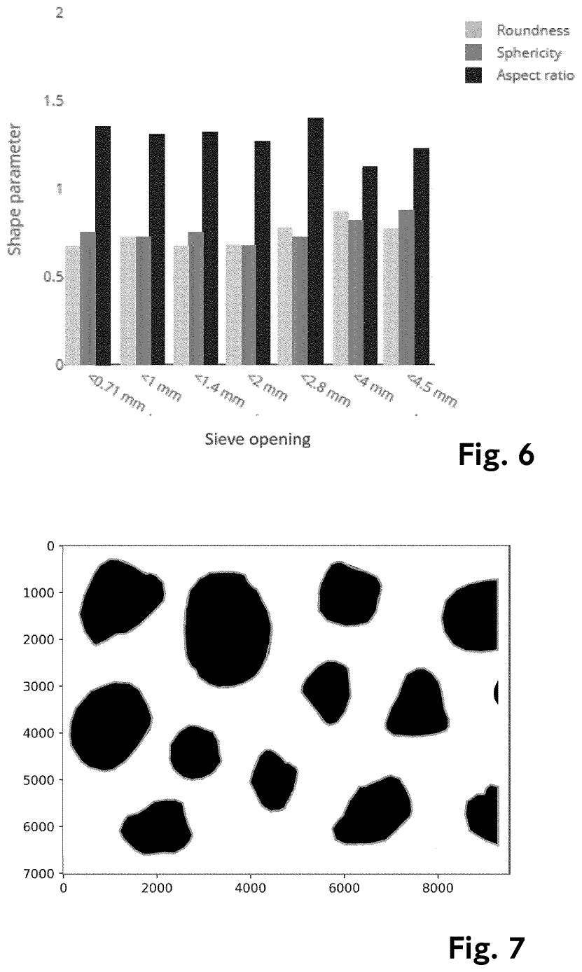

Furthermore, the file 50 comprises a two-dimensional plot 55 indicating the mean particle shape (for example roundness or sphericity) with a marker 55a. Additionally, the file 50 comprises a bar plot 57 displaying the mean value of selected particle shape parameters (for example roundness, sphericity and aspect ratio) per particle sieve size fraction. A more detailed view of the bar plot 57 is shown in FIG. 6. Of, course, the content of the data file 50 shown in FIG. 5 and the bar plot shown in FIG. 6 can be adapted to the specific sample and/or information required.

FIG. 7 shows schematic representation of an outline image overlaid over the corresponding digital image. The outline image can be used in addition to check the quality of the digital image and/or the analysis performed.

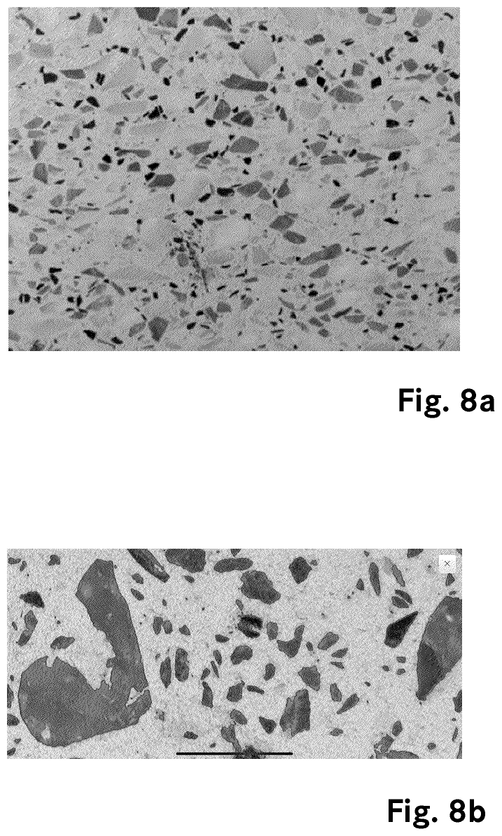

FIG. 8a shows a photography of the surface of a concrete floor comprising aggregates of different sizes and shapes that was analyzed with the inventive method to obtain information about the structure of the floor, e.g. the ratio of cement to coarse aggregate, the mix design analysis, the area share of the cementitious phase, and the quality of the production (effectiveness of vibrating; assessed on the basis of the homogeneity of aggregate distribution).

FIG. 8b shows a magnified section of FIG. 8a whereby, the identified particle shaped aggregates have been outlined.

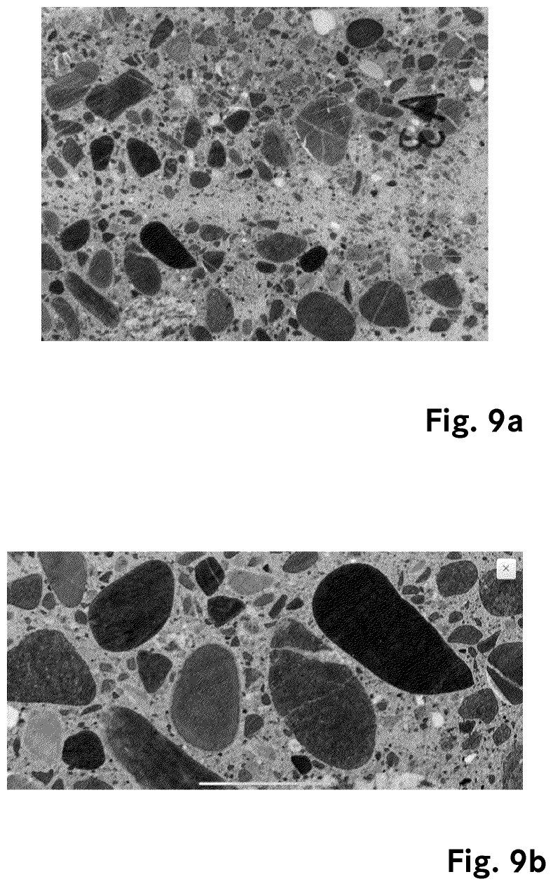

FIG. 9a shows a photography of the polished surface of a concrete drill core that was analyzed with the inventive method to check the mix design of the concrete. As can be seen from the photography, there is an inhomogeneous aggregate distribution, that can be quantified with the inventive method by determining the spatial distribution of the particle shaped constituents. Also, the ratio of cement to coarse aggregate, the mix design, and/or the binder share can be analyzed. When combined with information about the application method, it is for example possible to judge if the method was performed properly, e.g. with respect to the sufficiency of compaction by vibration.

FIG. 9b shows a magnified section of FIG. 9a whereby, the identified particle shaped aggregates have been outlined.

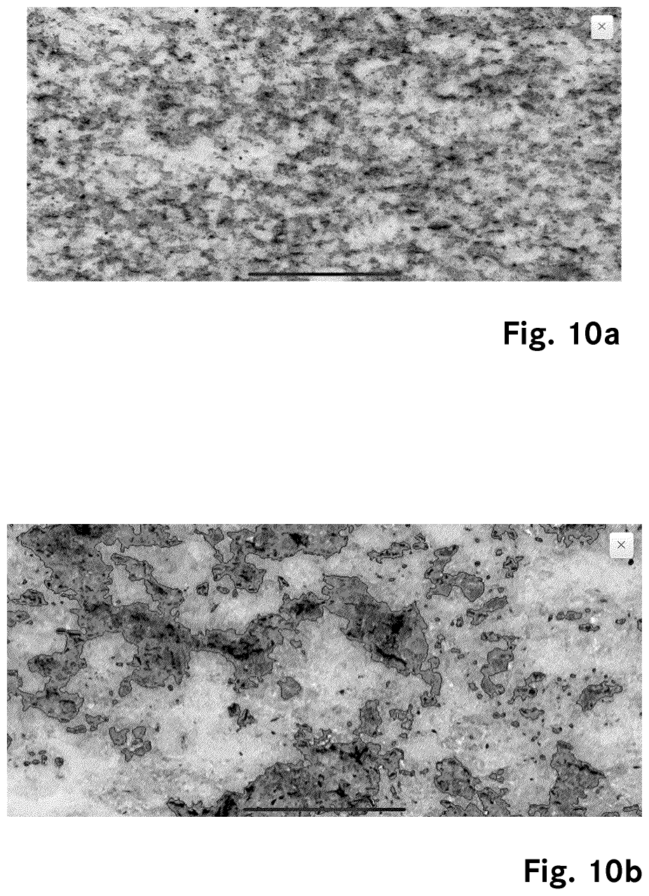

FIG. 10a shows a photography of a polished surface of granite that was analyzed with the inventive method to check the mineral composition or the amount of critical mineral phases as for example biotite. In this case, the surface consists of 42% Orthoclase, 15% Plagioclase, 35% Quartz and 8 % Biotit.

FIG. 10b shows a magnified section of FIG. 10a whereby, the identified particle shaped mineral phases have been outlined.

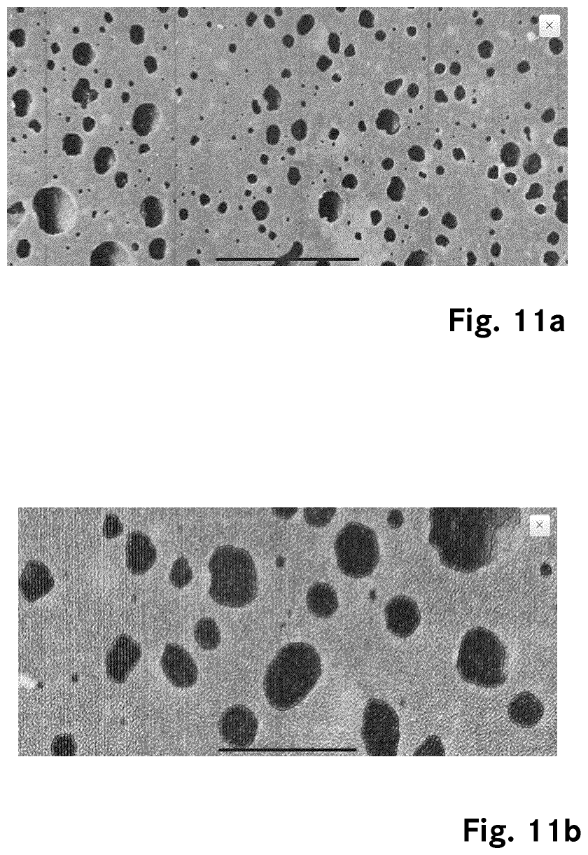

FIG. 11a shows a photography of a surface of an epoxy grout that was analyzed with the inventive method to identify the bearing area (area share of continuous phase) and the quantity of the air voids (particle shaped holes).

FIG. 11b shows a magnified section of FIG. 11a whereby, the identified air voids have been outlined.

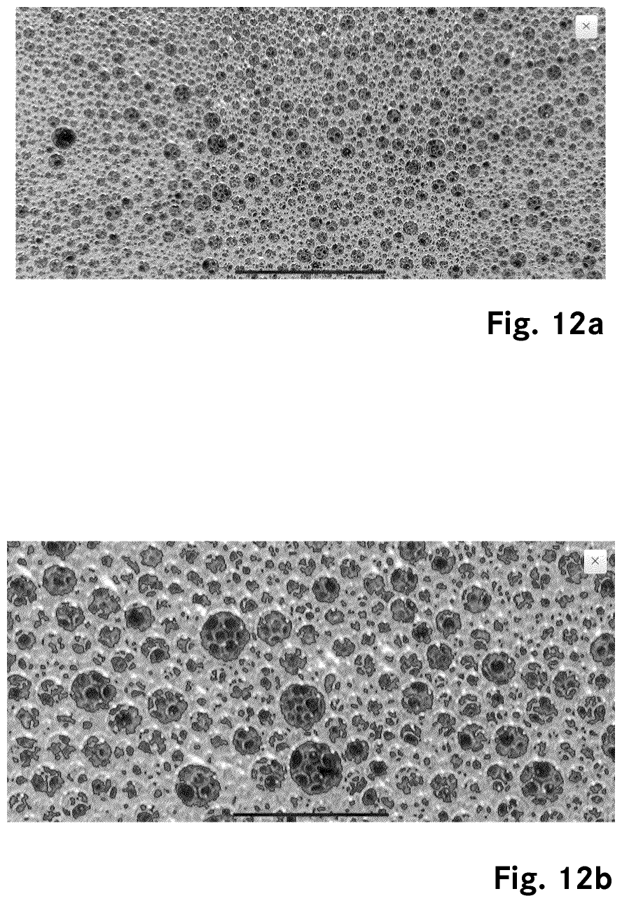

FIG. 12a shows a photography of a surface of a liquid aqueous foam that was analyzed with the inventive method to quantify the air content in the foam.

FIG. 12b shows a magnified section of FIG. 12a whereby, the identified air voids have been outlined.

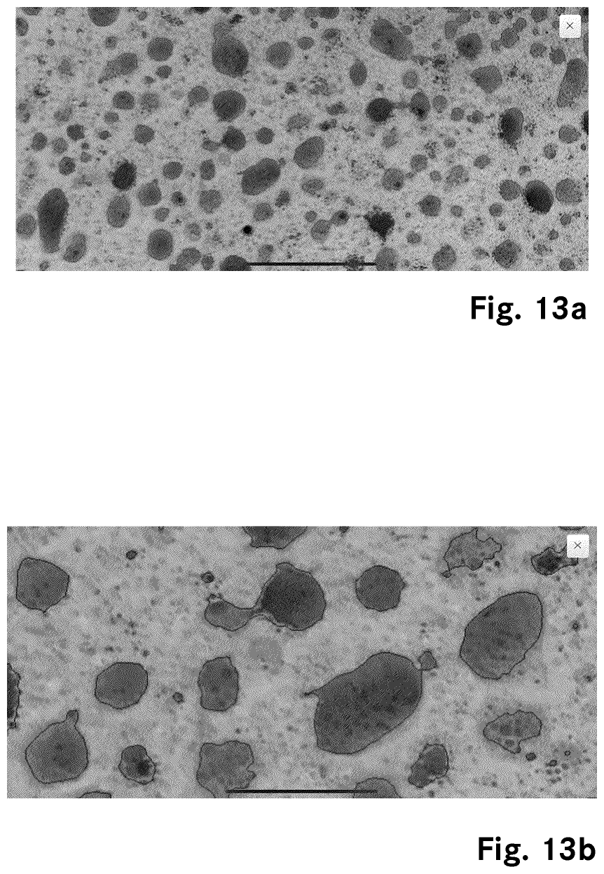

FIG. 13a shows a photography of a hardened foam that was analyzed with the inventive method to quantify the air content and the bearing area of the foam.

FIG. 13b shows a magnified section of FIG. 13a whereby, the identified air voids have been outlined.

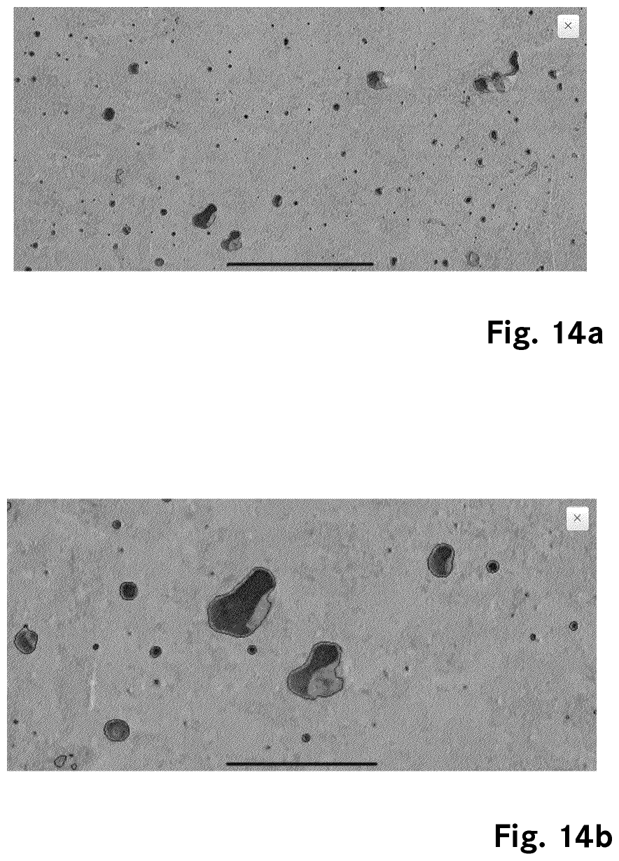

FIG. 14a shows a photography of a concrete wall with bugholes that was analyzed with the inventive method to check the quality of the wall (assessed by quantifying the area share of the bugholes).

FIG. 14b shows a magnified section of FIG. 14a whereby, the identified bugholes have been outlined.

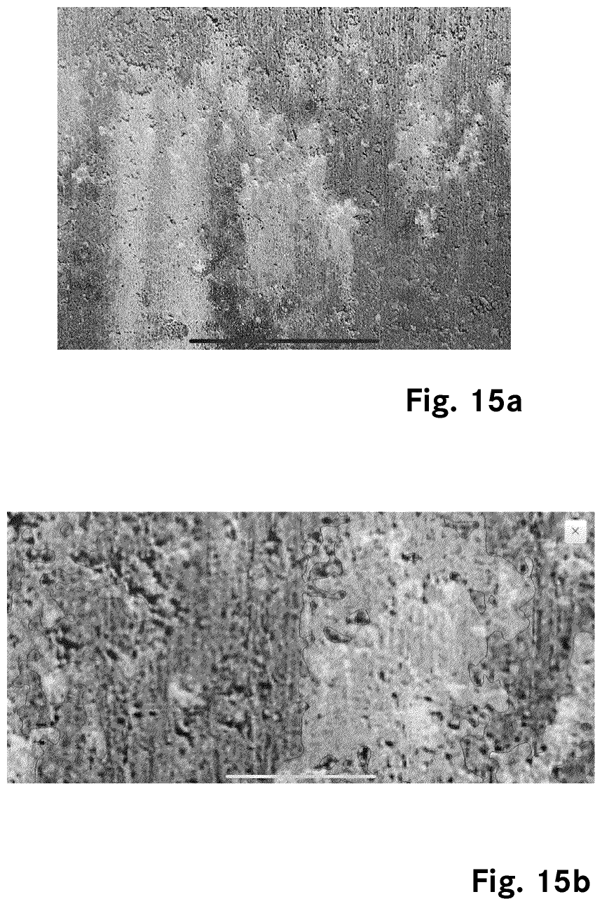

FIG. 15a shows a photography of a concrete wall with discoloration caused by efflorescence (bright area). The area share of the discolored region was quantified. This allows for example for determining the age of the concrete wall.

FIG. 15b shows a magnified section of FIG. 15a whereby, the discoloration region has been outlined.

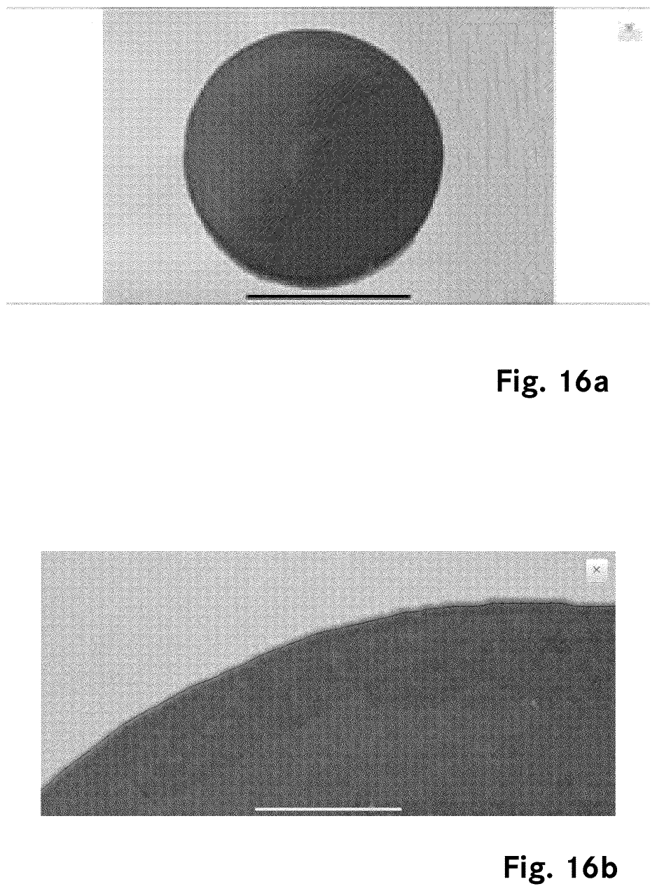

FIG. 16a shows a photography taken during a flow table test of a freshly prepared mortar sample. With the inventive method, the size of the mortar sample (dark round area) at a given time can be measured in order to obtain workability and/or consistence of the composition. FIG. 16b shows a magnified section of FIG. 16a.

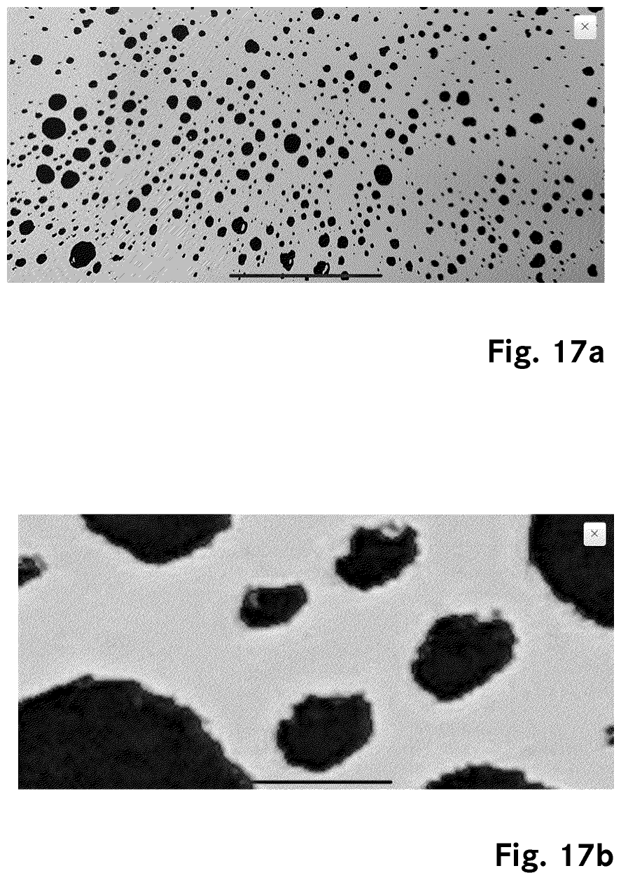

FIG. 17a shows a photography of a mortar sample with air voids that was inverted for determining the share of the bearing area and the air voids with the inventive method. FIG. 17b shows a magnified section of FIG. 17a whereby the identified air voids have been outlined.

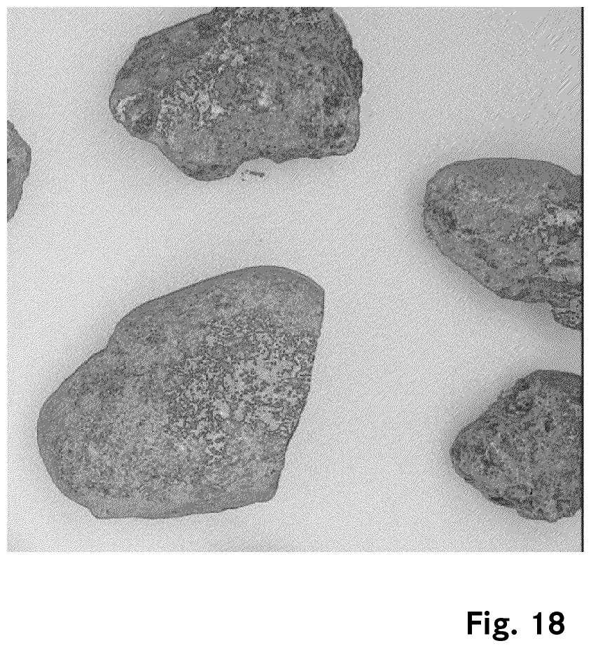

FIG. 18 shows a photography of several recycled aggregates with residues of cement (bright areas). With the inventive method, the quantity of binder present on recycled aggregates can be determined in order to judge the aggregate quality.

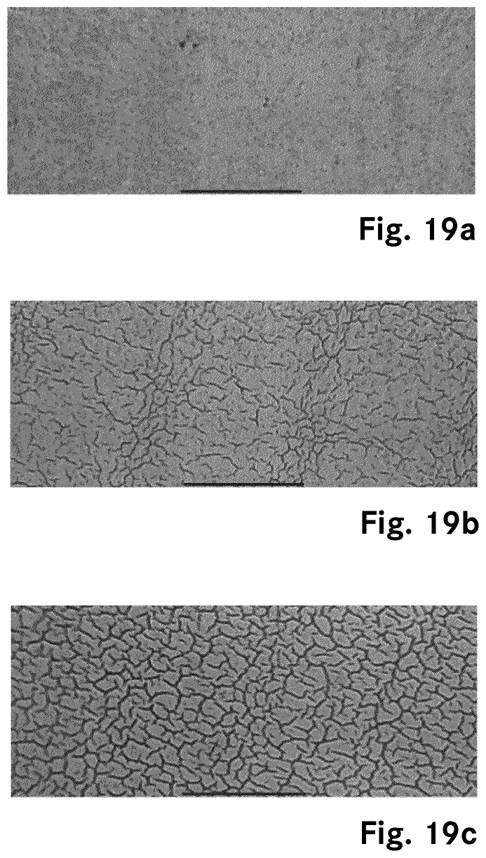

FIG. 19a-c shows photographs of the surfaces of three different synthetic membranes in different conditions, whereby the photographs were obtained with a camera with a magnification lens. In FIG. 19a, the membrane surface comprises only a surface texture but no cracks (no cracks; class 0 according to on EN 13956:2013). In FIG. 19b, the membrane surface comprises unbranched as well as branched cracks (class 1 according to on EN 13956:2013). In FIG. 19c, the membrane surface comprises a high density of predominantly branched cracks (class 2 according to on EN 13956:2013). With the inventive method, for example, the area share of the cracks can be determined with respect to surface of the membranes in order to determine the condition of the actual of the membranes. This allows e.g. for determining fracture types and classes directly from the photographs.

It will be appreciated by those skilled in the art that the present invention can be implemented in other specific forms without departing from the spirit or essential characteristics thereof. The presently disclosed implementations and embodiments are therefore considered in all respects to be illustrative and not restricted.

For example, instead of using server 21a, the application 26 can be configured as a standalone application capable of executing all of the steps 11, 12, 13, 14 and optionally 15a of the method of FIG. 1.

Likewise, it is possible to omit optional functions of the system 20, e.g. sharing of data with other computer devices, or adding additional functions, such as for example automatically retrieving of positional data via positioning sensors.

Also, instead or in addition to the particle size parameter and the particle shape parameter, a spatial distribution, a particle orientation parameter, a particle surface parameter, a particle roughness parameter, a packing density, a segregation parameter, a color, and/or an area share of the particle-shaped constituents can determined in step 13.
The particle size distribution of aggregates in the heterogeneous material;
The particle shape of aggregates in the heterogeneous material;
The quantity of a binder share, especially a paste share, in the heterogeneous material;
The quantity of binder attached to aggregates, e.g. in mortar or concrete materials;
A mix ratio of the heterogeneous material with respect to the particle-shaped constituents and the continuous phase;
A ration of coarse aggregates to cement (ca/c), e.g. in mortar or concrete materials;
The distribution of aggregates in the heterogeneous material;
The share, size and/or distribution of air voids in the heterogeneous material;
The quantity and/or quality of the mineral composition in the heterogeneous material;
The quality of the production method and/or special treatments received during production, for example the quality of compaction (e.g. vibration) and/or the quality of the application method;
The color distribution at the surface of the heterogeneous material, e.g. by characterizing carbonization and/or efflorescence for determining ageing of the sample;
The determination of a failure mode of the heterogeneous material;
Monitoring of mechanical defects in the heterogeneous material, e.g. cracks;
Workability related information, especially rheological properties, e.g. flow properties, slump flow, viscosity, t50 time, yield stress and/or consistency class; e.g. by taking one or more pictures of the sample in processable state, e.g. of the slump flow of the sample, at a predefined time or at predefined time intervals;
Predictive modelling, in particular the prediction of adjustments of the sample, e.g. raw material and/or mix design adjustments, especially for improving certain properties of the sample.

In FIG. 3, instead of or additionally to placing the mortar sample 31 in the sample area 30, similar to FIG. 4, a reference scale can be placed and/or marked on top of the sample 31, e.g. a ruler, a geometrical shape and/or or a letter code. In this case, it is sufficient to take a picture with a smaller image section. As long as the reference scale is visible in the picture, the size of particle shape constituents can be determined.

Also, it is possible to omit any reference scale and/or sample area. Thereby, it is possible manually set the dimension in the computer device, if desired. Noteworthy, even without any reference scale or dimensions, it is still possible to determine for example a share of the continuous phase and/or the homogeneity of the sample.

Furthermore, the structure of the data file 50 shown in FIG. 5 can be in any other file format and/or the respective information can be presented in a graphical user interface, e.g. a dashboard.

The method can as well be used for characterizing other samples, e.g. other suspensions, emulsions, foams, adhesives, air voids in walls and ceilings, aesthetic surface characteristics, etc.

1. A computer-implemented method for characterization of a heterogeneous material comprising distributed constituents, distributed particle-shaped constituents, dispersed within a continuous phase of condensed matter, comprising the steps of:
a) providing or selecting a sample of the heterogeneous material to be analyzed;
b) taking at least one digital image of the sample with a camera of a computer device, a mobile computer device, or a camera connected to a computer device, a mobile device and/or with the computer device read in at least one digital image that was pre-recorded with a standalone camera;
c) performing an image analysis, an imaging particle analysis, of the at least one digital image for extracting one or more of the following properties:
a size parameter, a particle size parameter, a shape parameter, a particle shape parameter, a spatial distribution, an orientation parameter, a particle orientation parameter, a surface parameter, a particle surface parameter, a roughness parameter, particle roughness parameter, a packing density, a segregation parameter, a color, and/or an area share of the constituents, the particle-shaped constituents, identified by the image analysis in the at least one digital image; and/or
an area share of the continuous phase identified by the image analysis in the at least one digital image;
d) making available the one or more of the properties extracted in step c) via a user interface, via a machine interface and/or on a data storage medium.

2. The method according to claim 1, whereby the distributed constituents, the particle-shaped constituents, are optically distinguishable from the continuous phase.

3. The method according to claim 1, whereby the distributed constituents, the particle-shaped constituents, have different light absorption and/or light reflection properties than the continuous phase, with respect to light having a wavelength in the range of 200nm-5′000 nm, in the range of 380-780 nm.

4. The method according to claim 1, whereby the heterogeneous material is a hardened binder material, hardened mineral binder composition or a hardened organic binder composition.

5. The method according to claim 1, whereby the heterogeneous material is a hardened concrete composition, a hardened mortar composition, or a hardened grout composition.

6. The method according to claim 1, whereby the heterogeneous material is an emulsion, a foam or a suspension.

7. The method according to claim 1, whereby the heterogeneous material is a synthetic material, made from polyvinyl chloride (PVC), and/or thermoplastic polyolefin (TPO), e.g. from the group comprising high-density polyethylene (HDPE), medium-density polyethylene (MDPE), low-density polyethylene (LDPE), polyethylene (PE), polyethylene terephthalate (PET), polystyrene (PS), polyvinyl chloride (PVC), polyamides (PA), ethylene/vinyl acetate copolymer (EVA), chlorosulfonated polyethylene, thermoplastic polyolefin elastomer (TPO, TPE-O), ethylene propylene diene rubber (EPDM), and mixtures thereof.

8. The method according to claim 1, whereby the constituents are distributed particle-shaped constituents in the form of solid particles.

9. The method according to claim 1, whereby the constituents are distributed particle-shaped constituents in the form of gas-filled pores, bugholes (surface air voids).

10. The method according to claim 1, whereby the distributed constituents are cracks, whereby the cracks are gas-filled.

11. (canceled)

12. The method according to claim 1, whereby the continuous phase comprises hardened binder material.

13. (canceled)

14. The method according to claim 1, whereby the constituents, the distributed particle-shaped constituents, comprise a first type of mineral material and the continuous phase of condensed matter comprises as second type of a mineral material, which is different from the first type of mineral material.

15. The method according to claim 1, whereby in step b) the at least one digital image is taken from a surface, of the sample of the heterogeneous material.

16. (canceled)

17. The method according to claim 1, whereby the sample or the flat surface is subjected to a surface treatment to increase a contrast between the continuous phase and the constituents, whereby, for example, the surface treatment is selected from coloring with an ink and/or polishing with a powder and/or a paste.

18. (canceled)

19. The method according to claim 1, whereby the mobile computer device is selected from a mobile phone, a mobile computer or a portable computer, and/or a head-mounted display with camera.

20. The method according to claim 1, whereby the camera is a camera for taking images in the visible spectrum, color images.

21.-23. (canceled)

24. The method according to claim 1, whereby when taking the image, the camera is aligned so that a share of the sample in the image is maximized, by providing alignment instructions to the user and/or by automatically adjusting at least one setting of the camera, e.g. the focal length of the camera.

25. The method according to claim 1, whereby a minimum detectable size, a minimum detectable particle size, of the constituents, the particle-shaped constituents, is calculated by taking into account the resolution of the camera, the length share of the sample in the total area of the image, and the real length of the sample.

26. The method according to claim 1, whereby, if a minimum detectable size, particle size, is below a predetermined threshold, a warning is provided to the user, alignment instructions are provided to the user and/or a setting of the camera, e.g. the focal distance, is adjusted automatically.

27. The method according to claim 1, whereby for each of the at least one digital image, an outline image, an inverted image and/or a color thresholded image is generated, and used as the image in step c).

28. The method according to claim 27, whereby the outline image, the inverted image and/or the color thresholded image is made available in step d) via a user interface, via a machine interface and/or on a data storage medium.

29. The method according to claim 1, whereby in step b), at least two, digital images are taken and for each image an imaging analysis is performed in step c), and by taking into account each of the one or more properties, the particle size parameter and/or the particle shape parameter, individually extracted from the at least two images, a deviation, the standard deviation, of the one or more properties, the particle size parameter and/or the at least one particle shape parameter, is determined.

30. The method according to claim 29, whereby, if the deviation is above a predetermined threshold, a warning is provided to the user and/or whereby a digital image and/or an outline image giving rise to diverging parameters is identified and/or indicated.

31.-37. (canceled)

38. The method according to claim 1, whereby the area share of the continuous phase is extracted, in particular to determine the binder share in the sample of the heterogeneous material.

39. (canceled)

40. The method according to claim 1, whereby the method is performed to obtain one or more of the following characteristics of the heterogeneous material, a hardened binder composition or a synthetic material:
the particle size distribution of aggregates in the heterogeneous material;
the particle shape of aggregates in the heterogeneous material;
the quantity of a binder share, a paste share, in the heterogeneous material;
the quantity of binder attached to aggregates, e.g. in mortar or concrete materials;
a mix ratio of the heterogeneous material with respect to the particle-shaped constituents and the continuous phase;
a ration of coarse aggregates to cement (ca/c), e.g. in mortar or concrete materials;
the distribution of aggregates in the heterogeneous material;
the share, size and/or distribution of air voids in the heterogeneous material;
the quantity and/or quality of the mineral composition in the heterogeneous material, whereby the quality is measured by determining the share of one or more particle shaped mineral constituents;
the quality of the production method and/or special treatments received during production, for example the quality of compaction (e.g. vibration) and/or the quality of the application method whereby the quality is measured by determining the homogeneity of the sample and/or the distribution of particle shaped constituents;
the color distribution at the surface of the heterogeneous material, e.g. for characterizing carbonization and/or efflorescence, for determining ageing of the sample;
the determination of a failure mode of the heterogeneous material, by measuring the number, size, shape and/or orientation of cracks in the sample;
monitoring of mechanical defects in the heterogeneous material, e.g. cracks;
an area share of cracks with respect to the area of the continuous phase, in a synthetic material, in a synthetic membrane;
a size, shape, spatial distribution, orientation, packing density, and/or segregation of cracks, in a synthetic material, in a synthetic membrane;
workability related information, rheological properties, e.g. flow properties, slump flow, viscosity, t50 time, yield stress and/or consistency class; e.g. by taking one or more pictures of the sample in processable state, e.g. of the slump flow of the sample, at a predefined time or at predefined time intervals;
predictive modelling, the prediction of adjustments for producing further samples, e.g. adjustment of raw material(s) and/or mix design adjustments, for improving certain properties of the further sample.

41. The method according to claim 1, whereby the method is at least partly, performed on the computer device.

42. The method according to claim 1, whereby the image analysis in step c) and/or the making available in step d) is/are conducted on a separate computer device, e.g. on a server.

43. The method according to claim 1, whereby the image, together with the at least one attribute, and the outline image, inverted image and/or thresholded image, is stored on an external computer device, e.g. a server, for sharing, recalling and/or further evaluation.

44. A system comprising a computer device, mobile computer device, and an additional separate computer device, whereby the system comprises:
(i) means for carrying out the steps a) to d), of the method of claim 1, and/or
(i) means for carrying out at least the steps a) and b), of the method of claim 1, and means for transferring at least one digital image of the sample, together with at least one attribute, to the separate computer device.

45. A system comprising a computer device, whereby the system comprises means for receiving at least one digital image of a sample of the heterogeneous material to be analyzed and means for carrying out steps c) and/or d) of the method of claim 1.

46. A computer-readable medium comprising instructions which, when executed by a computer device, a mobile computer device, causes the computer device to carry out steps a) to d), of the method of claim 1.

47. A computer-readable medium comprising instructions which, when executed by a computer device, cause the external computer device to receive at least one
digital image and perform steps c) and/or d) of the method of claim 1.
